(19) United States
(12) Patent Application Publication
(10) Pub. No.: US 2023/0391883 A1
(43) Pub. Date: Dec. 7, 2023

(54) Methods of Cancer Treatment Using Anti-TIGIT Antibodies in Combination with Anti-PD1 Antibodies

(71) Applicant: Beigene Switzerland GMBH, Basel (CH)

(72) Inventors: Tong Zhang, Beijing (CN); Liu Xue, Beijing (CN); Qi Liu, Beijing (CN); Min Wei, Beijing (CN); Kang Li, Beijing (CN); Yunxia Zuo, Beijing (CN)

(21) Appl. No.: 18/262,236

(22) PCT Filed: Jan. 20, 2022
(86) PCT No.: PCT/CN2022/072869

§ 371 Date: Jul. 20, 2023

(30) Foreign Application Priority Data
Jan. 21, 2021 (WO) PCT/CN2021/073083
Jan. 22, 2021 (WO) PCT/CN2021/073308
Jan. 28, 2021 (WO) PCT/CN2021/074141
Feb. 1, 2021 (WO) PCT/CN2021/074644
Feb. 4, 2021 (WO) PCT/CN2021/075310
May 5, 2021 (WO) PCT/CN2021/091822
Jul. 29, 2021 (WO) PCT/CN2021/109322

(51) Int. Cl. C07K 16/28; A61P 35/00

(52) U.S. Cl. CPC C07K 16/2896; C07K 16/2818; A61P 35/00; A61K 2039/507

(57) ABSTRACT

Provided are methods of treating cancer or increasing, enhancing, or stimulating an immune response with antibodies that specifically bind to TIGIT (T cell immunoreceptor with Ig and ITIM domains, WUCAM or Vstm3) and antigen-binding fragments thereof in combination with an anti-PD1 antibody.

### FIELD

The present application relates to antibodies that specifically bind to TIGIT (T cell immunoreceptor with Ig and ITIM domains) in combination with anti-PD1 antibodies for the treatment of cancer.

### BACKGROUND

TIGIT (T cell immunoglobulin and ITIM domain) is a type I transmembrane protein, a member of the CD28 family of proteins that plays an important role in inhibiting T- and NK cell-mediated functional activities in anti-tumor immunity (Boles K S, et al., 2009 Eur J Immunol, 39:695-703; Stanietsky N, et al., 2009 PNAS 106:17858-63; Yu X, et al. 2009 Nat. Immunol, 10:48-57).

The genes and cDNAs coding for TIGIT were cloned and characterized in mouse and human. Full length human TIGIT has a sequence of 244 amino acids (SEQ ID NO: 1) in length, in which the first 21 amino acids consist a signal peptide. The amino acid sequence of the mature human TIGIT contains 223 amino acid (aa) residues (NCBI accession number: NM_173799). The extracellular domain (ECD) of mature human TIGIT consists of 120 amino acid residues (SEQ ID NO: 2, corresponding to amino acids 22-141 of SEQ ID NO: 1) with a V-type Ig-like domain (corresponding to amino acids 39-127 of SEQ ID NO: 1), followed by a 21 aa transmembrane sequence, and an 82 aa cytoplasmic domain with an immunoreceptor tyrosine-based inhibitory motif (ITIM) (Yu X, et al. 2009 Nat. Immunol, 10:48-57; Stengel K F, et al. 2012 PNAS 109:5399-04). Within the ECD, human TIGIT shares only 59% and 87% aa sequence identity with mouse and cynomolgus monkey, respectively.

TIGIT is expressed on T cells (including activated T cells, memory T cells, regulatory T (Treg) cells, and follicular T helper (Tfh) cells), and NK cells (Boles K S, et al., 2009 Eur J Immunol, 39:695-703; Joller N, et al., 2014 Immunity 40:569-81; Levin S D, et al., 2011 Eur J Immunol, 41:902-15; Stanietsky N, et al., 2009 PNAS 106:17858-63; Yu X, et al. 2009 Nat. Immunol, 10:48-57).

So far, two TIGIT ligands, CD155 (also known as poliovirus receptor or PVR) and CD112 (also known as poliovirus receptor-related 2, PVRL2, nectin-2), have been identified. These ligands are primarily expressed on APCs (such as dendritic cells and macrophages) and tumor cells (Casado J G, et al., 2009 Cancer Immunol Immunother 58:1517-26; Levin S D, et al., 2011 Eur J Immunol, 41:902-15; Mendelsohn C L et al., 1989 56:855-65; Stanietsky N, et al., 2009 PNAS 106:17858-63; Yu X, et al. 2009 Nat. Immunol, 10:48-57). As an immune “checkpoint” molecule, TIGIT initiates inhibitory signaling in immune cells when engaged by its ligands, CD155 and CD112. The binding affinity of TIGIT to CD155 (Kd: ˜1 nM) is much higher than to CD112 and whether the TIGIT: CD112 interaction is functionally relevant in mediating inhibitory signals yet remain to be determined. A co-stimulatory receptor, CD226 (DNAM-1), binds to the same ligands with lower affinity (Kd: ˜100 nM), but delivers a positive signal (Bottino C, et al., 2003 J Exp Med 198:557-67). In addition, CD96 (Tactile), a “TIGIT-like” receptor, also plays a similarly inhibitory role in the same pathway (Chan C J, et al., 2014 Nat. Immunol 15:431-8).

TIGIT can inhibit immune responses through different mechanisms. First, interaction between TIGIT and PVR on dendritic cells (DCs) could deliver a “reverse signaling” in DCs, leading to up-regulation of IL-10 and decrease of IL-12 secretion, thereby inhibiting T-cell activation (Yu X, et al. Nat Immunol. 2009 10:48-57). Second, TIGIT binds to CD155 with higher affinity, thereby competing off DNAM-1-CD155 interaction. Third, direct ligation of TIGIT on T cells could down-regulate TCR-mediated activation and subsequent proliferation and engagement of TIGIT on NK cells block NK cell cytotoxicity (Joller N, et al. 2011 186: 1338-42; Stanietsky N, et al., 2009 PNAS 106:17858-63). Fourth, TIGIT expression on Tregs has been associated with a highly activated and suppressive phenotype in tumor tissue and TIGIT signaling in Tregs may favor Treg stability (Joller N, et al. Immunity 2014 40:569-81; Kurtulus S, et al. J Clin Invest. 2015 125: 4053-4062).

TIGIT has an immunoglobulin tail tyrosine (ITT)-like motif followed by an immunoreceptor tyrosine-based inhibition motif (ITIM) in its cytoplasmic tail (Yu X, et al. Nat Immunol. 2009 10:48-57; Engels N, et al. Curr Opin Immunol 2011 23: 324-329). These motifs could mediate recruitment of the phosphatase SHIP-1 and β-arrestin 2 (Li M, et al. J Biol Chem. 2014 289:17647-17657; Liu S, et al. Cell death and differentiation 2013 20: 456-464), thus providing a mechanism by which TIGIT can intrinsically deliver inhibitory signals to dampen activating signals.

Up-regulation of TIGIT expression in tumor-infiltrating lymphocytes (TILs) and peripheral blood mononuclear cells (PBMCs) has been reported in many types of cancers such as lung (Tassi, et al., Cancer Res. 2017 77: 851-861), esophageal (Xie J, et al., Oncotarget 2016 7:63669-63678), breast (Gil Del Alcazar C R, et al. 2017 Cancer Discov.), acute myeloid leukemia (AML) (Kong Y et al., Clin Cancer Res. 2016 22:3057-66) and melanoma (Chauvin J M, et al., J Clin Invest. 2015 125:2046-2058). The increased expression of TIGIT in AML is associated with poor prognosis of patient survival outcome (Kong Y et al., Clin Cancer Res. 2016 22:3057-66). Not only does up-regulation of TIGIT signaling play important roles in immune tolerance to cancer, but also to chronic viral infection. During HIV infection, expression of TIGIT on T cells was significantly higher and positively correlated with viral loads and disease progression (Chew G M, et al., 2016 PLoS Pathog. 12:e1005349). In addition, blockade of TIGIT receptor alone or in combination with other blockade could rescue functionally “exhausted” T cells both in vitro and in vivo (Chauvin J M, et al., J Clin Invest. 2015 125:2046-2058; Chew G M, et al., 2016 PLoS Pathog. 12:e1005349; Johnston R J, et al. Cancer Cell 2014 26:923-937). In the cases of cancer and viral infections, activation of TIGIT signaling promotes immune cell dysfunction, leading to the cancer outgrowth or extended viral infection. Inhibition of TIGIT-mediated inhibitory signaling by therapeutic agents may restore the functional activities of immune cells including T cells, NK cells and dendritic cells (DCs), therefore enhancing immunity against cancer or chronic viral infection.

Monoclonal antibodies that target either PD1 or PDL1, can block this interaction and boost the immune response against cancer cells. These antibodies have been shown to be helpful in treating several types of cancer, including melanoma of the skin, non-small cell lung cancer (NSCLC), kidney cancer, bladder cancer, head and neck cancers, and Hodgkin lymphoma. Cancer cells in most non-responders to single-agent checkpoint inhibitors escape through innate mechanisms that allow the cancer cells to grow and survive. As a result, disease progresses at a rate consistent with the natural history. However, unlike intrinsic resistance, late relapses are now emerging in patients with prior clinical benefit after longer follow-up of clinical trials, suggesting the emergence of acquired resistance (Jenkins et al., Br. J. Cancer 118, 9-16 2018).

Therefore, the combination of anti-TIGIT antibodies in combination with anti-PD1 antibodies can rescue immune cells from tolerance, inducing efficient immune responses in the treatment of cancer or chronic viral infections.

### SUMMARY OF THE INVENTION

The present disclosure is directed methods of cancer treatment, administering anti-TIGIT antibodies in combination with an anti-PD1 antibody.

In particular embodiments, the anti-TIGIT antibody or an antigen-binding fragment comprises a heavy chain variable region (VH) comprising one, two or three CDRs having an amino acid sequence selected from SEQ ID NOs: 3, 4, 5 or 13, or variants thereof comprising one or more conservative substitutions, e.g. one or two conservative substitutions in the amino acid sequences of SEQ ID NOs 3, 4, 5 or 13; and/or a light chain variable region (VL) comprising one, two or three CDRs having an amino acid sequence selected from SEQ ID NOs: 6, 7, or 8, or variants thereof comprising one or more conservative substitutions, e.g. one or two conservative substitutions in the amino acid sequences of SEQ ID NOs: 6, 7, or 8.

In a more specific embodiment, the anti-TIGIT antibody or an antigen-binding fragment thereof comprises a heavy chain variable region (VH) comprising a VH-CDR1 having an amino acid sequence of SEQ ID NO: 3 or a variant thereof comprising one or more conservative substitutions, e.g. one or two conservative substitutions, a VH-CDR2 having an amino acid sequence of SEQ ID NO: 4 or SEQ ID NO: 13 or a variant thereof comprising one or more conservative substitutions, e.g. one or two conservative substitutions, and a VH-CDR3 having an amino acid sequence of SEQ ID NO: 5 or a variant thereof comprising one or more conservative substitutions, e.g. one or two conservative substitutions; and/or a light chain variable region (VL) comprising a VL-CDR1 having an amino acid sequence of SEQ ID NO: 6 or a variant thereof comprising one or more conservative substitutions, e.g. one or two conservative substitutions, a VL-CDR2 having an amino acid sequence of SEQ ID NO: 7 or a variant thereof comprising one or more conservative substitutions, e.g. one or two conservative substitutions, and a VL-CDR3 having an amino acid sequence of SEQ ID NO: 8 or a variant thereof comprising one or more conservative substitutions, e.g. one or two conservative substitutions.

The anti-TIGIT antibody or an antigen-binding fragment thereof of the present application is capable of binding to human TIGIT and comprises a heavy chain variable region having an amino acid sequence selected from SEQ ID NO: 9, 14, 19, or a sequence having at least 85%, at least 90%, at least 95%, at least 96%, at least 97%, at least 98%, at least 99% sequence identity with SEQ ID NO: 9, 14, 19. In one embodiment, the difference in sequence lies in the framework region. In one embodiment, the antibody or the antigen-binding fragment thereof comprises a heavy chain variable region encoded by a nucleotide sequence selected from SEQ ID NO: 10, 15 or 20, or a variant thereof.

The antibody or the antigen-binding fragment thereof of the present application is capable of binding to human TIGIT and comprises a heavy chain variable region having an amino acid sequence selected from SEQ ID NO: 11, 16, 21, or 24, or a sequence having at least 85%, at least 90%, at least 95%, at least 96%, at least 97%, at least 98%, at least 99% sequence identity with SEQ ID NO: 11, 16, 21 or 24. In one embodiment, the difference in sequence lies in the framework region. In one embodiment, the antibody or the antigen-binding fragment thereof comprising a heavy chain variable region encoded by an nucleotide sequence selected from SEQ ID NO: 12, 17 or 22, or a variant thereof.

In one embodiment, the antibody or antigen-binding fragment thereof is capable of binding to human TIGIT with a Kd value of about 1×10⁻⁹ M to about 1×10⁻¹² M. For example, the antibody or the antigen-binding fragment thereof is capable of binding to human TIGIT with a Kd value less than about 1×10⁻⁹ M, less than about 1×10⁻¹⁰ M, less than about 1×10⁻¹¹ M, or less than about 1×10⁻¹² M.

In one embodiment, the antibody or the antigen-binding fragment thereof comprises a heavy chain constant region of the subclass of IgG1, IgG2, IgG3, or IgG4 or a variant thereof, and a light chain constant region of the type of kappa or lambda or a variant thereof. In a more specific embodiment, the Fc region of the antibody is human IgG1 Fc or a variant thereof, e.g. a Fc region of SEQ ID NO: 18.

A method of cancer treatment, the method comprising administering to a subject an effective amount of an anti-TIGIT antibody or antigen-binding fragment thereof in combination with an anti-PD1 antibody or antigen binding fragment thereof.

The method, wherein the method comprises administering to a subject an effective amount of an antibody or antigen-binding fragment thereof, which specifically binds to human TIGIT and comprises:
(i) a heavy chain variable region that comprises (a) a HCDR (Heavy Chain Complementarity Determining Region) 1 of SEQ ID NO: 3, (b) a HCDR2 of SEQ ID NO: 4, and (c) a HCDR3 of SEQ ID NO:5; and a light chain variable region that comprises (d) a LCDR (Light Chain Complementarity Determining Region) 1 of SEQ ID NO:6, (e) a LCDR2 of SEQ ID NO:7, and (f) a LCDR3 of SEQ ID NO:8; or
(ii) a heavy chain variable region that comprises (a) a HCDR1 of SEQ ID NO:3, (b) a HCDR2 of SEQ ID NO:13, and (c) a HCDR3 of SEQ ID NO:5; and a light chain variable region that comprises: (d) a LCDR1 of SEQ ID NO:6, (e) a LCDR2 of SEQ ID NO:7, and (f) a LCDR3 of SEQ ID NO: 8; in combination with an anti-PD1 antibody.

The method, wherein the TIGIT antibody or antigen-binding fragment thereof comprises:
(i) a heavy chain variable region (VH) that comprises SEQ ID NO:19, and a light chain variable region (VL) that comprises SEQ ID NO: 21;
(ii) a heavy chain variable region (VH) that comprises SEQ ID NO: 14, and a light chain variable region (VL) that comprises SEQ ID NO: 16; or
(iii) a heavy chain variable region (VH) that comprises SEQ ID NO: 9, and a light chain variable region (VL) that comprises SEQ ID NO: 11.

The method, wherein the anti-PD1 antibody comprises an antibody or an antigen binding fragment thereof which specifically binds human PD1, and comprises:
a heavy chain variable region that comprises (a) a HCDR1 of SEQ ID NO: 25, (b) HCDR2 of SEQ ID NO: 26, and (c) HCDR3 of SEQ ID NO: 27; and a light chain variable region that comprises (d) LCDR1 of SEQ ID NO: 28, (e) LCDR2 of SEQ ID NO: 29, and (f) LCDR3 of SEQ ID NO: 30.

The method, wherein the anti-PD1 antibody or antigen binding fragment thereof which specifically binds human PD1, and comprises a heavy chain variable region (VH) comprising an amino acid sequence of SEQ ID NO:32 and a light chain variable region (VL) comprising an amino acid sequence of SEQ ID NO: 33.

The method wherein the anti-PD1 antibody comprises an IgG4 constant domain comprising SEQ ID NO: 35.

The method, wherein the anti-TIGIT antibody is an antibody fragment selected from the group consisting of Fab, Fab′-SH, Fv, scFv, and (Fab′)2 fragments.

The method, wherein the anti-PD1 antibody is an antibody fragment selected from the group consisting of Fab, Fab′-SH, Fv, scFv, and (Fab′)2 fragments.

The method, wherein the cancer is selected from the group consisting of breast cancer, colon cancer, pancreatic cancer, head and neck cancer, gastric cancer, kidney cancer, liver cancer, small cell lung cancer, non-small cell lung cancer, ovarian cancer, skin cancer, mesothelioma, lymphoma, leukemia, myeloma or sarcoma.

The method, wherein the small cell lung cancer is limited stage small cell lung cancer.

The method, wherein the cancer is non-small cell lung cancer.

The method, wherein the head and neck cancer is nasopharyngeal cancer.

The method, wherein the esophageal cancer is esophageal squamous cell carcinoma (ESCC).

The method, wherein the cancer is uterine cancer.

The method, wherein the gastric cancer is gastric or Gastroesophageal Junction Cancer.

The method, wherein the cervical cancer is recurring or metastatic cervical cancer.

The method, wherein the skin cancer is basal cell carcinoma.

The method, wherein the cancer is pancreatic cancer.

The method, further comprising the administration of chemotherapy.

The method, wherein the chemotherapy is chemoradiotherapy.

The method wherein the anti-PD1 antibody is dosed at 200 mg every three weeks.

The method wherein the anti-TIGIT antibody is dosed at a range of 50 mg-900 mg.

The method wherein the anti-TIGIT antibody is dosed at 50 mg every three weeks.

The method wherein the anti-TIGIT antibody is dosed at 150 mg every three weeks.

The method wherein the anti-TIGIT antibody is dosed at 450 mg every three weeks.

The method wherein the anti-TIGIT antibody is dosed at 900 mg every three weeks. The method wherein the anti-TIGIT antibody is dosed at 1800 mg every three weeks.

### BRIEF DESCRIPTION OF THE DRAWINGS

FIG. 1 Schematic diagram of TIGIT-mIgG2a (top) and TIGIT-huIgG1 (bottom). TIGIT ECD: TIGIT extracellular domain. N: N-terminus. C: C-terminus.

FIG. 2A-B Phylogenetic trees of anti-TIGIT antibody Vh (A) and Vk (B) regions. The Vh and Vk sequences of candidate anti-TIGIT antibodies were aligned using DNASTAR's Megalign software. Sequence homology was displayed in phylogenetic trees.

FIG. 3 Affinity determination of purified murine anti-TIGIT antibodies by surface plasmon resonance (SPR).

FIG. 4A-B Determination of TIGIT binding by flow cytometry.

FIG. 5A-B (A) A schematic diagram showing the inhibition of TIGIT-ligand interactions by anti-TIGIT mAbs. (B) The binding of soluble TIGIT (TIGIT-huIgG1 fusion protein) to TIGIT ligand-expressing HEK293 cells (HEK293/PVR or HEK293/PVR-L2) was determined by flow cytometry. The blockade of TIGIT-ligand interaction was quantitatively measured by adding serially diluted anti-TIGIT antibodies. Results were shown in mean±SD of duplicates.

FIG. 6A-B Activation of CMV-specific human T cells by anti-TIGIT mAbs. Human CMV peptide (NLVPMVATV, 495-503)-sensitized HLA-A2.1⁺ PBMCs (4×10⁴) were stimulated with the CMV peptide-pulsed target cells HCT116 cells (10⁴) overnight in the presence of anti-TIGIT antibodies. IFN-γ in the culture supernatant was determined by ELISA. All conditions were performed in triplicates. Results were shown as mean±SD.

FIG. 7A-B Anti-TIGIT mAbs promote NK cell-mediated cytotoxicity. (A) TIGIT and DNAM-1 expression on engineered NK92MI/TIGIT-DNAM-1 stable cell line. (B) Killing of NK92MI/TIGIT-DNAM-1 cells against SK-MES-1/PVR cells in the presence of hu1217-2-2/IgG1mf (0.007-30 μg/ml) was determined by an lactate dehydrogenase (LDH) release assay as described in Example 8. Results are shown in mean±SD of triplicates.

FIG. 8 Anti-TIGIT mAb hu1217-2-2/IgG1wt reduces the surface expression of TIGIT receptor via FcγR-mediated trogocytosis. Jurkat/TIGIT cells were incubated with Fc γ R-expressing HEK293 cells in the presence of biotin-labeled anti-TIGIT mAbs in complete media overnight. In some cases, 10% human AB serum was added to determine the effects of bulk human IgG on trogocytosis. Surface expression of TIGIT receptor was determined by staining with SA-APC (Biolegend). MFI was determined by flow cytometry. All data points were in duplicates. Results were shown in mean±SD.

FIG. 9A shows ADCC effects of anti-TIGIT mAbs on human peripheral blood mononuclear cells (PBMCs). TIGIT expression on PHA-stimulated PBMCs from healthy donors was determined by flow cytometry. CD4⁺ (CD4⁺Foxp3⁻), CD8⁺ T effectors and regulatory T cells (Tregs, CD4⁺ Foxp3⁺) all expressed significant levels of TIGIT (18˜41%). Data shown are representative results from 3 healthy donors.

FIG. 9B is ADCC assay was performed using a CD16⁺ human NK cell line NK92MI/CD16V as effector cells and PHA-stimulated PBMCs as target cells in the presence of TIGIT mAbs (30 μg/mL) or control antibodies (OKT3 at 5 μg/ml as a positive control, and huIgG at 30 μg/ml as a negative control) for 42 hrs. Percentages of CD3⁺, CD8⁺ T cells and Tregs were determined by flow cytometry.

FIG. 10 demonstrates CDC effects of anti-TIGIT mAbs on human PBMCs. CDC assay was performed using PHA-stimulated PBMCs as target cells and autologous sera as the source of complements. After 3 days of co-culture of pre-activated PBMCs with anti-TIGIT mAbs (0.01-100 μg/ml) in the final concentration of 15% autologous sera, percentage of CDC (y-axis) was measured by cell-titer glow assay and calculated as described in Example 11. Data from donors A and B are shown. HuIgG were used as a negative control, whereas anti-MHC-A, B, C was used as a positive control.

FIG. 11 depicts the decline of serum concentrations of hu1217-2-2 after intravenous infusion from 50 mg to 900 mg.

FIG. 12 shows that hu1217-2-2 either as a single agent or in combination with BGB-A317 promotes IFN-γ secretion in vitro.

FIG. 13 demonstrates that hu1217-2-2 can reduce tumor growth as a single agent in a mouse glioma model.

FIG. 14 shows the efficacy of hu1217-2-2 in combination with an anti-PD1 antibody in a TIGIT knock in mouse model of MC colon cancer.

### DETAILED DESCRIPTION

### Definitions

Conservative amino acid substitutions of amino acids are commonly known in the art and exemplarily shown in the table below. Generally, a conservative amino acid substitution means that an amino acid residue is replaced by another amino acid residue having a similar side chain.

| Original amino | One-letter and | Conservative |
|---|---|---|
| acid residue | three-letter codes | substitution(s) |
| Alanine | A or Ala | Gly; Ser |
| Arginine | R or Arg | Lys; His |
| Asparagine | N or Asn | Gln; His |
| Aspartic acid | D or Asp | Gln; Asn |
| Cysteine | C or Cys | Ser; Ala |
| Glutamine | Q or Gln | Asn |
| Glutamic acid | E or Glu | Asp; Gln |
| Glycine | G or Gly | Ala |
| Histidine | H or His | Asn; Gln |
| Isoleucine | I or Ile | Leu; Val |
| Leucine | L or Leu | Ile; val |
| Lysine | K or Lys | Arg; His |
| Methionine | M or Met | Leu; Ile; Tyr |
| Phenylalanine | F or Phe | Tyr; Met; Leu |
| Proline | P or Pro | Ala |
| Serine | S or Ser | Thr |
| Threonine | T or Thr | Ser |
| Tryptophan | W or Trp | Tyr; Phe |
| Tyrosine | Y or Tyr | Trp; Phe |
| Valine | V or Val | Ile; Leu |

Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the” include their corresponding plural references unless the context clearly dictates otherwise.

The term “or” is used to mean, and is used interchangeably with, the term “and/or” unless the context clearly dictates otherwise.

Throughout this specification and the claims which follow, unless the context requires otherwise, the word “comprise”, and variations such as “comprises” and “comprising”, will be understood to imply the inclusion of a stated amino acid sequence, DNA sequence, step or group thereof, but not the exclusion of any other amino acid sequence, DNA sequence, step. When used herein the term “comprising” can be substituted with the term “containing”, “including” or sometimes “having.”

The term “TIGIT” includes various mammalian isoforms, e.g., human TIGIT, orthologs of human TIGIT, and analogs comprising at least one epitope within TIGIT. The amino acid sequence of TIGIT, e.g., human TIGIT, and the nucleotide sequence encoding the same, is known in the art (see Genbank AAI01289).

The terms “administration,” “administering,” “treating” and “treatment” as used herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” or “treatment” also includes in vitro and ex vivo treatments, e.g., of a cell, by a reagent, diagnostic, binding compound, or by another cell. The term “subject” herein refers to any organism, preferably an animal, more preferably a mammal (e.g., rat, mouse, dog, cat, rabbit) and most preferably a human.

The term “antibody” herein is used in the broadest sense and specifically covers antibodies (including full length monoclonal antibodies) and antibody fragments so long as they recognize antigen, e.g., TIGIT. An antibody is usually monospecific, but may also be described as idiospecific, heterospecific, or polyspecific. Antibody molecules bind by means of specific binding sites to specific antigenic determinants or epitopes on antigens.

The term “monoclonal antibody” or “mAb” or “Mab” herein means a population of substantially homogeneous antibodies, i.e., the antibody molecules comprised in the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their complementarity determining regions (CDRs), which are often specific for different epitopes. The modifier “monoclonal” indicates the character of the antibody as being obtained from a substantially homogeneous population of antibodies and is not to be construed as requiring production of the antibody by any particular method. Monoclonal antibodies (mAbs) may be obtained by methods known to those skilled in the art. See, for example Kohler G et al., Nature 1975 256:495-497; U.S. Pat. No. 4,376,110; Ausubel F M et al., CURRENT PROTOCOLS IN MOLECULAR BIOLOGY 1992; Harlow E et al., ANTIBODIES: A LABORATORY MANUAL, Cold spring Harbor Laboratory 1988; and Colligan J E et al., CURRENT PROTOCOLS IN IMMUNOLOGY 1993. The mAbs disclosed herein may be of any immunoglobulin class including IgG, IgM, IgD, IgE, IgA, and any subclass thereof. A hybridoma producing a mAb may be cultivated in vitro or in vivo. High titers of mAbs can be obtained by in vivo production where cells from the individual hybridomas are injected intraperitoneally into mice, such as pristine-primed Balb/c mice to produce ascites fluid containing high concentrations of the desired mAbs. MAbs of isotype IgM or IgG may be purified from such ascites fluids, or from culture supernatants, using column chromatography methods well known to those of skill in the art.

In general, the basic antibody structural unit comprises a tetramer. Each tetramer includes two identical pairs of polypeptide chains, each pair having one “light chain” (about 25 kDa) and one “heavy chain” (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as α, δ, ε, γ, or μ, and define the antibody's isotypes as IgA, IgD, IgE, IgG, and IgM, respectively. Within light and heavy chains, the variable and constant regions are joined by a “J” region of about 12 or more amino acids, with the heavy chain also including a “D” region of about 10 more amino acids.

The variable regions of each light/heavy chain (VL/VH) pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called “complementarity determining regions (CDRs)”, which are located between relatively conserved framework regions (FR). The CDRs are usually aligned by the framework regions, enabling binding to a specific epitope. In general, from N-terminal to C-terminal, both light and heavy chain variable domains sequentially comprise FR-1 (or FR1), CDR-1 (or CDR1), FR-2 (FR2), CDR-2 (CDR2), FR-3 (or FR3), CDR-3 (CDR3), and FR-4 (or FR4). The assignment of amino acids to each domain is, generally, in accordance with the definitions of Sequences of Proteins of Immunological Interest, Kabat, et al., National Institutes of Health, Bethesda, Md.; 5th ed.; NIH Publ. No. 91-3242 (1991); Kabat (1978) Adv. Prot. Chem. 32: 1-75; Kabat, et al., (1977) J. Biol. Chem. 252:6609-6616; Chothia, et al, (1987) J Mol. Biol. 196:901-917 or Chothia, et al., (1989) Nature 342:878-883.

The term “hypervariable region” means the amino acid residues of an antibody that are responsible for antigen-binding. The hypervariable region comprises amino acid residues from a “CDR” (i.e., VL-CDR1, VL-CDR2 and VL-CDR3 in the light chain variable domain and VH-CDR1, VH-CDR2 and VH-CDR3 in the heavy chain variable domain). See, Kabat et al. (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). The term “framework” or “FR” residues mean those variable domain residues other than the hypervariable region residues defined herein as CDR residues.

Unless otherwise indicated, “antibody fragment” or “antigen-binding fragment” means antigen binding fragments of antibodies, i.e., antibody fragments that retain the ability to bind specifically to the antigen bound by the full-length antibody, e.g. fragments that retain one or more CDR regions. Examples of antigen binding fragments include, but not limited to, Fab, Fab′, F(ab′)2, and Fv fragments; diabodies; linear antibodies; single-chain antibody molecules, e.g., single chain Fv (ScFv); nanobodies and multispecific antibodies formed from antibody fragments.

An antibody that binds to a specified target protein with specificity is also described as specifically binding to a specified target protein. This means the antibody exhibits preferential binding to that target as compared to other proteins, but this specificity does not require absolute binding specificity. An antibody is considered “specific” for its intended target if its binding is determinative of the presence of the target protein in a sample, e.g., without producing undesired results such as false positives. Antibodies or binding fragments thereof, useful in the present invention will bind to the target protein with an affinity that is at least two fold greater, preferably at least 10-times greater, more preferably at least 20-times greater, and most preferably at least 100-times greater than the affinity with non-target proteins. An antibody herein is said to bind specifically to a polypeptide comprising a given amino acid sequence, e.g., the amino acid sequence of a mature human TIGIT molecule, if it binds to polypeptides comprising that sequence but does not bind to proteins lacking that sequence.

The expressions “pH-dependent binding”, “pH-dependent target binding” and “pH-dependent antigen binding” are interchangeable in the present disclosure, indicating that the antibody of the present application binds to its target/antigen, namely human TIGIT, in a pH-dependent manner. Specifically, the antibody of the present application shows a higher binding affinity and/or binding signal to its antigen at a mild acidic pH, e.g. pH 6.0, which is usually found in tumor microenvironment, as compared to the binding affinity and/or binding signal at physiologic pH, e.g. pH 7.4. The methods for determining the binding affinity and/or the intensity of binding signal of the antibody of the present application are well known in the art and include but not limited to surface plasmon resonance (Biacore) or similar technology. More specifically, the antibody of the present application has a K_{D} ratio at pH 7.4/pH 6.0 of greater than 2, 3, 4, 5, 6, 7, 8, 9, 10, 20, 30, 40, 50, 60, 70, 80, 90, 100, or more, as measured by surface plasmon resonance (Biacore) or similar technology. Alternatively, or additionally, the antibody of the present application has a Rmax (RU) value at pH 6.0 which is at least 2-fold, 3-fold, 4-fold, 5-fold, 6-fold, 7-fold, 8-fold, 9-fold, 10-fold, 20-fold, 30-fold, 40-fold, 50- fold higher than the Rmax at pH 7.4 as measured by surface plasmon resonance (Biacore) or similar technology. The binding affinity of the antibody can be measured at 25° C. or 37° C. Tumor microenvironment has been found to show a relatively more acidic pH than physiological condition or normal tissues (Zhang et al. Focus on molecular Imaging 2010; Tannock and Rotin et al. Cancer Res 1989). Therefore, the antibody of the present application having above-mentioned pH-dependent binding is advantageous as an anti-TIGIT therapeutic agent for targeting TIGIT-positive lymphocytes in the tumor microenvironment with selectivity and having lower toxicity associated with periphery activation of lymphocytes.

The term “human antibody” herein means an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” means an antibody that comprises only mouse or rat immunoglobulin protein sequences, respectively.

The term “humanized antibody” means forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu”, “Hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

The antibody of the present application has potential therapeutic uses in treating cancer. The term “cancer” or “tumor” herein means or describes the physiological condition in mammals that is typically characterized by unregulated cell growth. Examples of cancer include but are not limited to, lung cancer (including small-cell lung cancer, or non-small cell lung cancer), adrenal cancer, liver cancer, stomach cancer, cervical cancer, melanoma, renal cancer, breast cancer, colorectal cancer, leukemia, bladder cancer, bone cancer, brain cancer, an endometrial cancer, head and neck cancer, lymphoma, ovarian cancer, skin cancer, thyroid tumor, or metastatic lesion of the cancer.

Further, the antibody of the present application has potential therapeutic uses in controlling viral infections and other human diseases that are mechanistically involved in immune tolerance or “exhaustion.” In the context of the present application, the term “exhaustion” refers to a process which leads to a depleted ability of immune cells to respond during to a cancer or a chronic viral infection.

The term “therapeutically effective amount” as herein used, refers to the amount of an antibody that, when administered to a subject for treating a disease or a disorder, or at least one of the clinical symptoms of a disease or disorder, is sufficient to affect such treatment for the disease, disorder, or symptom. The “therapeutically effective amount” can vary with the antibody, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the active agents comprised in the combination for the effective treatment of a disease, a disorder or a condition.

The “subject” as used herein is a mammal, e.g., a rodent or a primate, preferably a higher primate, e.g., a human (e.g., a patient having, or at risk of having, a disorder described herein).

### Anti-TIGIT Antibodies

The present disclosure provides for antibodies, antigen-binding fragments, that specifically bind human TIGIT. Furthermore, the present disclosure provides antibodies that have desirable pharmacokinetic characteristics and other desirable attributes, and thus can be used for reducing the likelihood of or treating cancer. The present disclosure further provides pharmaceutical compositions comprising the antibodies and methods of making and using such pharmaceutical compositions for the prevention and treatment of cancer and associated disorders.

The present disclosure provides for antibodies or antigen-binding fragments thereof that specifically bind to TIGIT. Antibodies or antigen-binding fragments of the present disclosure include, but are not limited to, the antibodies or antigen-binding fragments thereof, generated as described, below.

The present disclosure provides antibodies or antigen-binding fragments that specifically bind to TIGIT, wherein said antibodies or antibody fragments (e.g., antigen-binding fragments) comprise a VH domain having an amino acid sequence of SEQ ID NO:9, 14 or 19. The present disclosure also provides antibodies or antigen-binding fragments that specifically bind TIGIT, wherein said antibodies or antigen-binding fragments comprise a VH CDR having an amino acid sequence of any one of the VH CDRs provided herein. In one aspect, the present disclosure provides antibodies or antigen-binding fragments that specifically bind to TIGIT, wherein said antibodies comprise (or alternatively, consist of) one, two, three, or more VH CDRs having an amino acid sequence of any of the VH CDRs provided in the present disclosure.

The present disclosure provides for antibodies or antigen-binding fragments that specifically bind to TIGIT, wherein said antibodies or antigen-binding fragments comprise a VL domain having an amino acid sequence of SEQ ID NO:11, 16 or 21. The present disclosure also provides antibodies or antigen-binding fragments that specifically bind to TIGIT, wherein said antibodies or antigen-binding fragments comprise a VL CDR having an amino acid sequence of any one of the VL CDRs listed herein. In particular, the disclosure provides for antibodies or antigen-binding fragments that specifically bind to TIGIT, said antibodies or antigen-binding fragments comprise (or alternatively, consist of) one, two, three or more VL CDRs having an amino acid sequence of any of the VL CDRs of the current disclosure.

Other antibodies or antigen-binding fragments thereof of the present disclosure include amino acids that have been mutated, yet have at least 60%, 70%, 80%, 90%, 95% or 99% percent identity in the CDR regions with the CDR regions depicted in the sequences described herein. In some aspects, it includes mutant amino acid sequences wherein no more than 1, 2, 3, 4 or 5 amino acids have been mutated in the CDR regions when compared with the CDR regions disclosed in the sequences provided.

Other antibodies of the present disclosure include those where the amino acids or nucleic acids encoding the amino acids have been mutated; yet have at least 60%, 70%, 80%, 90%, 95% or 99% percent identity to the sequences described in Table 5. In some aspects, it includes mutant amino acid sequences wherein no more than 1, 2, 3, 4 or 5 amino acids have been mutated in the variable regions when compared with the variable regions depicted in the sequence described herein, while retaining substantially the same therapeutic activity.

### Anti-PD1 Antibodies

The present disclosure provides for anti-PD1 antibodies found, for example, in U.S. Pat. No. 8,735,553. PD1 antibodies are also provided herein and comprise, for example, a heavy chain variable region (VH) comprising the complementarity determining regions (CDRs): HCDR1 as set forth in SEQ ID NO: 25, HCDR2 as set forth in SEQ ID NO: 26, and HCDR3 as set forth in SEQ ID NO: 27; and a light chain variable region (VL) comprising: LCDR1 as set forth in SEQ ID NO:28, LCDR2 as set forth in SEQ ID NO: 29, and LCDR3 as set forth in SEQ ID NO: 30. This antibody is designated herein as “BGB-A317.”

In another embodiment, the anti-PD1 antibody or antigen-binding fragment which specifically binds human PD1 and comprises a heavy chain variable region (VH) comprising an amino acid sequence of SEQ ID NO:32 and a light chain variable region (VL) comprising an amino acid sequence of SEQ ID NO: 34. In yet another embodiment, the anti-PD1 antibody comprises an IgG4 constant domain comprising SEQ ID NO: 35.

### Further Alteration of the Framework of Fc Region

In yet other aspects, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in, e.g., U.S. Pat. Nos. 5,624,821 and 5,648,260, both by Winter et al.

In another aspect, one or more amino acid residues can be replaced with one or more different amino acid residues such that the antibody has altered C1q binding and/or reduced or abolished complement dependent cytotoxicity (CDC). This approach is described in, e.g., U.S. Pat. No. 6,194,551 by Idusogie et al.

In yet another aspect, one or more amino acid residues are altered to thereby alter the ability of the antibody to fix complement. This approach is described in, e.g., the PCT Publication WO 94/29351 by Bodmer et al. In a specific aspect, one or more amino acids of an antibody or antigen-binding fragment thereof of the present disclosure are replaced by one or more allotypic amino acid residues, for the IgG1 subclass and the kappa isotype. Allotypic amino acid residues also include, but are not limited to, the constant region of the heavy chain of the IgG1, IgG2, and IgG3 subclasses as well as the constant region of the light chain of the kappa isotype as described by Jefferis et al., MAbs. 1:332-338 (2009).

In another aspect, the Fc region is modified to increase the ability of the antibody to mediate antibody dependent cellular cytotoxicity (ADCC) and/or to increase the affinity of the antibody for an Fcγ receptor by modifying one or more amino acids. This approach is described in, e.g., the PCT Publication WO 00/42072 by Presta. Moreover, the binding sites on human IgG1 for FcγRI, FcγRII, FcγRIII and FcRn have been mapped and variants with improved binding have been described (see Shields et al., J. Biol. Chem. 276:6591-6604, 2001).

In still another aspect, the glycosylation of an antibody is modified. For example, an aglycosylated antibody can be made (i.e., the antibody lacks or has reduced glycosylation). Glycosylation can be altered to, for example, increase the affinity of the antibody for “antigen.” Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation can increase the affinity of the antibody for antigen. Such an approach is described in, e.g., U.S. Pat. Nos. 5,714,350 and 6,350,861 by Co et al.

Additionally, or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Lecl3 cells, with reduced ability to attach fucose to Asn (297)-linked carbohydrates, also resulting in hypofucosylation of antibodies expressed in that host cell (see also Shields et al., (2002) J. Biol. Chem. 277:26733-26740). PCT Publication WO 99/54342 by Umana et al., describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., Nat. Biotech. 17:176-180, 1999).

In another aspect, if a reduction of ADCC is desired, human antibody subclass IgG4 was shown in many previous reports to have only modest ADCC and almost no CDC effector function (Moore G L, et al. 2010 MAbs, 2:181-189). On the other hand, natural IgG4 was found less stable in stress conditions such as in acidic buffer or under increasing temperature (Angal, S. 1993 Mol Immunol, 30:105-108; Dall'Acqua, W. et al, 1998 Biochemistry, 37:9266-9273; Aalberse et al. 2002 Immunol, 105:9-19). Reduced ADCC can be achieved by operably linking the antibody to IgG4 engineered with combinations of alterations to have reduced or null FcγR binding or C1q binding activities, thereby reducing or eliminating ADCC and CDC effector functions. Considering physicochemical properties of antibody as a biological drug, one of the less desirable, intrinsic properties of IgG4 is dynamic separation of its two heavy chains in solution to form half antibody, which lead to bi-specific antibodies generated in vivo via a process called “Fab arm exchange” (Van der Neut Kolfschoten M, et al. 2007 Science, 317:1554-157). The mutation of serine to proline at position 228 (EU numbering system) appeared inhibitory to the IgG4 heavy chain separation (Angal, S. 1993 Mol Immunol, 30:105-108; Aalberse et al. 2002 Immunol, 105:9-19). Some of the amino acid residues in the hinge and γFc region were reported to have impact on antibody interaction with Fcγ receptors (Chappel S M, et al. 1991 Proc. Natl. Acad. Sci. USA, 88:9036-9040; Mukherjee, J. et al., 1995 FASEB J, 9:115-119; Armour, K. L. et al. 1999 Eur J Immunol, 29:2613-2624; Clynes, R. A. et al, 2000 Nature Medicine, 6:443-446; Arnold J. N., 2007 Annu Rev immunol, 25:21-50). Furthermore, some rarely occurring IgG4 isoforms in human population can also elicit different physicochemical properties (Brusco, A. et al. 1998 Eur J Immunogenet, 25:349-55; Aalberse et al. 2002 Immunol, 105:9-19). To generate TIGIT antibodies with low ADCC, CDC and instability, it is possible to modify the hinge and Fc region of human IgG4 and introduce a number of alterations. These modified IgG4 Fc molecules can be found disclosed in SEQ ID NOs: 83-88, U.S. Pat. No. 8,735,553.

### Antibody Production

Anti-TIGIT antibodies and antigen-binding fragments thereof can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

The disclosure further provides polynucleotides encoding the antibodies described herein, e.g., polynucleotides encoding heavy or light chain variable regions or segments comprising the complementarity determining regions as described herein. In some aspects, the polynucleotide encoding the heavy chain variable regions has at least 85%, 89%, 90%, 91%, 92%, 93%, 94%, 95%, 96%, 97%, 98%, 99%, or 100% nucleic acid sequence identity with a polynucleotide that encodes for the polypeptides of SEQ ID NOs: 9, 14 or 19. In some aspects, the polynucleotide encoding the light chain variable regions has at least 85%, 89%, 90%, 91%, 92%, 93%, 94%, 95%, 96%, 97%, 98%, 99%, or 100% nucleic acid sequence identity with a polynucleotide that encodes for the polypeptides of SEQ ID NOs:11, 16, or 21.

The polynucleotides of the present disclosure can encode the variable region sequence of an anti-TIGIT antibody. They can also encode both a variable region and a constant region of the antibody. Some of the polynucleotide sequences encode a polypeptide that comprises variable regions of both the heavy chain and the light chain of one of the exemplified anti-TIGIT antibodies. Some other polynucleotides encode two polypeptide segments that respectively are substantially identical to the variable regions of the heavy chain and the light chain of one of the murine antibodies.

Also provided in the present disclosure are expression vectors and host cells for producing the anti-TIGIT antibodies. The choice of expression vector depends on the intended host cells in which the vector is to be expressed. Typically, the expression vectors contain a promoter and other regulatory sequences (e.g., enhancers) that are operably linked to the polynucleotides encoding an anti-TIGIT antibody chain or antigen-binding fragment. In some aspects, an inducible promoter is employed to prevent expression of inserted sequences except under the control of inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements can also be required or desired for efficient expression of an anti-TIGIT antibody or antigen-binding fragment. These elements typically include an ATG initiation codon and adjacent ribosome binding site or other sequences. In addition, the efficiency of expression can be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer can be used to increase expression in mammalian host cells.

The host cells for harboring and expressing the anti-TIGIT antibody chains can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express anti-TIGIT polypeptides. Insect cells in combination with baculovirus vectors can also be used.

In other aspects, mammalian host cells are used to express and produce the anti-TIGIT polypeptides of the present disclosure. For example, they can be either a hybridoma cell line expressing endogenous immunoglobulin genes or a mammalian cell line harboring an exogenous expression vector. These include any normal mortal or normal or abnormal immortal animal or human cell. For example, a number of suitable host cell lines capable of secreting intact immunoglobulins have been developed, including the CHO cell lines, various COS cell lines, HEK 293 cells, myeloma cell lines, transformed B-cells and hybridomas. The use of mammalian tissue cell culture to express polypeptides is discussed generally in, e.g., Winnacker, From Genes to Clones, VCH Publishers, NY, N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters can be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

### Methods of Detection and Diagnosis

The antibodies or antigen-binding fragments of the present disclosure are useful in a variety of applications including, but not limited to, methods for the detection of TIGIT. In one aspect, the antibodies or antigen-binding fragments are useful for detecting the presence of TIGIT in a biological sample. The term “detecting” as used herein includes quantitative or qualitative detection. In certain aspects, a biological sample comprises a cell or tissue. In other aspects, such tissues include normal and/or cancerous tissues that express TIGIT at higher levels relative to other tissues.

In one aspect, the present disclosure provides a method of detecting the presence of TIGIT in a biological sample. In certain aspects, the method comprises contacting the biological sample with an anti-TIGIT antibody under conditions permissive for binding of the antibody to the antigen and detecting whether a complex is formed between the antibody and the antigen. The biological sample can include, without limitation, urine or blood samples.

Also included is a method of diagnosing a disorder associated with expression of TIGIT. In certain aspects, the method comprises contacting a test cell with an anti-TIGIT antibody; determining the level of expression (either quantitatively or qualitatively) of TIGIT in the test cell by detecting binding of the anti-TIGIT antibody to the TIGIT polypeptide; and comparing the level of expression in the test cell with the level of TIGIT expression in a control cell (e.g., a normal cell of the same tissue origin as the test cell or a non-TIGIT expressing cell), wherein a higher level of TIGIT expression in the test cell as compared to the control cell indicates the presence of a disorder associated with expression of TIGIT.

### Methods of Treatment

The antibodies or antigen-binding fragments of the present disclosure are useful in a variety of applications including, but not limited to, methods for the treatment of an TIGIT-associated disorder or disease. In one aspect, the TIGIT-associated disorder or disease is a cancer.

In one aspect, the present disclosure provides a method of treating cancer. In certain aspects, the method comprises administering to a patient in need an effective amount of an anti-TIGIT antibody or antigen-binding fragment. The cancer can include, without limitation, breast cancer, head and neck cancer, gastric cancer, kidney cancer, liver cancer, small cell lung cancer, non-small cell lung cancer, ovarian cancer, skin cancer, mesothelioma, lymphoma, leukemia, myeloma and sarcoma.

An antibody or antigen-binding fragment of the invention can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies or antigen-binding fragments of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody or antigen-binding fragment of the invention will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 100 mg/kg of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. Such doses can be administered intermittently, e.g., every week or every three weeks (e.g., such that the patient receives from about two to about twenty, or e.g. about six doses of the antibody). An initial higher loading dose, followed by one or more lower doses can be administered. However, other dosage regimens can be useful. The progress of this therapy is easily monitored by conventional techniques and assays.

### Combination Therapy

In one aspect, TIGIT antibodies of the present disclosure can be used in combination with other therapeutic agents, for example anti-PD1 antibodies. Other therapeutic agents that can be used with the TIGIT antibodies of the present disclosure include: but are not limited to, a chemotherapeutic agent (e.g., paclitaxel or a paclitaxel agent; (e.g. Abraxane®), docetaxel; carboplatin; topotecan; cisplatin; irinotecan, doxorubicin, lenalidomide, 5-azacytidine, ifosfamide, oxaliplatin, pemetrexed disodium, cyclophosphamide, etoposide, decitabine, fludarabine, vincristine, bendamustine, chlorambucil, busulfan, gemcitabine, melphalan, pentostatin, mitoxantrone, pemetrexed disodium), tyrosine kinase inhibitor (e.g., EGFR inhibitor (e.g., erlotinib), multikinase inhibitor (e.g., MGCD265, RGB-286638), CD-20 targeting agent (e.g., rituximab, ofatumumab, RO5072759, LFB-R603), CD52 targeting agent (e.g., alemtuzumab), prednisolone, darbepoetin alfa, lenalidomide, Bcl-2 inhibitor (e.g., oblimersen sodium), aurora kinase inhibitor (e.g., MLN8237, TAK-901), proteasome inhibitor (e.g., bortezomib), CD-19 targeting agent (e.g., MEDI-551, MOR208), MEK inhibitor (e.g., ABT-348), JAK-2 inhibitor (e.g., INCB018424), mTOR inhibitor (e.g., temsirolimus, everolimus), BCR/ABL inhibitor (e.g., imatinib), ET-A receptor antagonist (e.g., ZD4054), TRAIL receptor 2 (TR-2) agonist (e.g., CS-1008), HGF/SF inhibitor (e.g., AMG 102), EGEN-001, Polo-like kinase 1 inhibitor (e.g., BI 672).

TIGIT antibodies of the present disclosure can be used in combination with other therapeutics, for example, anti-PD1 antibodies. Anti-PD1 antibodies can include, without limitation, antibodies disclosed in U.S. Pat. No. 8,735,553. Pembrolizumab (formerly MK-3475), as disclosed by Merck, is a humanized IgG4-K immunoglobulin with a molecular weight of about 149 kDa, which targets the PD1 receptor and inhibits binding of the PD1 receptor ligands PD-L1 and PD-L2. Pembrolizumab has been approved for the indications of metastatic melanoma and metastatic non-small cell lung cancer (NSCLC) and is under clinical investigation for the treatment of head and neck squamous cell carcinoma (HNSCC), and refractory Hodgkin's lymphoma (cHL). Nivolumab (as disclosed by Bristol-Meyers Squibb is a fully human IgG4-K monoclonal antibody. Nivolumab (clone 5C4) is disclosed in U.S. Pat. No. US 8,008,449 and WO 2006/121 168. Nivolumab is approved for the treatment of melanoma, lung cancer, kidney cancer, and Hodgkin's lymphoma.

### Pharmaceutical Compositions and Formulations

Also provided are compositions, including pharmaceutical formulations, comprising an anti-TIGIT antibody or antigen-binding fragment, or polynucleotides comprising sequences encoding an anti-TIGIT antibody or antigen-binding fragment. In certain embodiments, compositions comprise one or more antibodies or antigen-binding fragments that bind to TIGIT, or one or more polynucleotides comprising sequences encoding one or more antibodies or antigen-binding fragments that bind to TIGIT. These compositions can further comprise suitable carriers, such as pharmaceutically acceptable excipients including buffers, which are well known in the art.

Pharmaceutical formulations of an TIGIT antibody or antigen-binding fragment as described herein are prepared by mixing such antibody or antigen-binding fragment having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3- pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Pat. No. 7,871,607 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

Sustained-release preparations can be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility can be readily accomplished, e.g., by filtration through sterile filtration membranes.

### Pharmaceutical Compositions and Kits

In some aspects, this disclosure provides compositions, e.g., pharmaceutically acceptable compositions, which include an anti-TIGIT antibody described herein, formulated together with at least one pharmaceutically acceptable excipient. As used herein, the term “pharmaceutically acceptable excipient” includes any and all solvents, dispersion media, isotonic and absorption delaying agents, and the like that are physiologically compatible. The excipient can be suitable for intravenous, intramuscular, subcutaneous, parenteral, rectal, spinal or epidermal administration (e.g. by injection or infusion).

The compositions herein may be in a variety of forms. These include, for example, liquid, semi-solid and solid dosage forms, such as liquid solutions (e.g., injectable and infusion solutions), dispersions or suspensions, liposomes, and suppositories. A suitable form depends on the intended mode of administration and therapeutic application. Typical suitable compositions are in the form of injectable or infusion solutions. One suitable mode of administration is parenteral (e.g., intravenous, subcutaneous, intraperitoneal, intramuscular). In some embodiments, the antibody is administered by intravenous infusion or injection. In certain embodiments, the antibody is administered by intramuscular or subcutaneous injection.

### EXAMPLE

### Example 1: Generation of Anti-TIGIT Monoclonal Antibody

Anti-TIGIT monoclonal antibodies (mAbs) were generated based on conventional hybridoma fusion technology (de St Groth and Sheidegger, 1980 J Immunol Methods 35:1; Mechetner, 2007 Methods Mol Biol 378:1) with minor modifications. The mAbs with high binding activity in enzyme-linked immunosorbent assay (ELISA) and fluorescence-activated cell sorting (FACS) assay were selected for further characterization.

### TIGIT Recombinant Protein for Immunization and Binding Assays

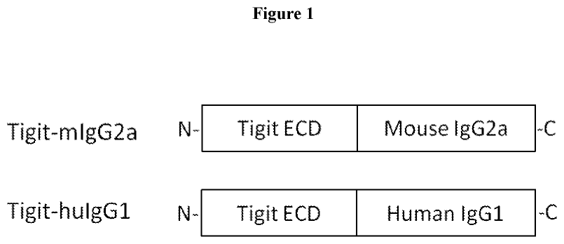

The cDNA coding for the full-length human TIGIT (SEQ ID NO:1) was synthesized by and purchased from Sino Biological (Beijing, China) based on its GenBank sequence (Accession No: NM_173799). The coding region of extracellular domain (ECD) of the full-length human TIGIT corresponding to the amino acid (AA) 1-141 of SEQ ID NO: 1 was PCR-amplified and cloned into pcDNA3.1-based expression vector (Invitrogen, Carlsbad, CA, USA) with C-terminus fused either to the Fc domain of mouse IgG2a or to the Fc domain of human IgG1 heavy chain, which resulted in two recombinant fusion protein expression plasmids, TIGIT-mIgG2a and TIGIT-huIgG1, respectively. The schematic presentation of TIGIT fusion proteins were shown in FIG. 1. For the recombinant fusion protein production, TIGIT-mIgG2a and TIGIT-huIgG1 plasmids were transiently transfected into 293G cells (developed in-house) and cultured for 7 days in a CO₂ incubator equipped with rotating shaker. The supernatant containing the recombinant protein was collected and cleared by centrifugation. TIGIT-mIgG2a and TIGIT-huIgG1 were purified using a Protein A column (Cat.: 17127901, GE Life Sciences). Both TIGIT-mIgG2a and TIGIT-huIgG1 proteins were dialyzed against phosphate buffered saline (DPBS) and stored in −80° C. freezer in small aliquots.

### Stable Expression Cell Lines

To establish stable cell lines that express full-length human TIGIT (huTIGIT) or monkey TIGIT (mkTIGIT, accession #: XM_005548101.2), TIGIT genes (synthesized by Genescript, Nanjing, China) were cloned into a retroviral vector pFB-Neo (Cat.: 217561, Agilent, USA). Dual-tropic retroviral vectors were generated according to a previous protocol (Zhang T, et al. 2005, Blood). Vectors containing huTIGIT and mkTIGIT were transduced into Jurkat and NK92MI cells (ATCC, Manassas, VA, USA), respectively, to generate the cell lines, Jurkat/huTIGIT and NK92MI/mkTIGIT. The high expression cell lines were selected by cultivation in medium with G418 and FACS binding assay.

### Immunization, Hybridoma Fusion and Cloning

Eight to twelve week-old Balb/c mice (from HFK BIOSCIENCE CO., LTD, Beijing, China) were immunized intraperitoneally (i.p.) with 100 μL, of antigen mixture containing 10 μg of TIGIT-mIgG2a and a water-soluble adjuvant (Cat.: KX0210041, KangBiQuan, Beijing, China). The procedure was repeated three weeks later. Two weeks after the 2^{nd} immunization, mouse sera were evaluated for TIGIT binding by ELISA and FACS. Ten days after serum screening, the mice with the highest anti-TIGIT antibody serum titers were boosted via i.p. injection with 50 μg of TIGIT-mIgG2a. Three days after boosting, the splenocytes were isolated and fused to the murine myeloma cell line, SP2/0 cells (ATCC), using the standard techniques (Gefter et al., Somat Cell Genet, 1977 3(2):231-6).

### Assessment of TIGIT Binding Activity of Antibodies by ELISA and FACS

The supernatants of hybridoma clones were initially screened by ELISA as described in Methods in Molecular Biology (2007) 378:33-52 with some modifications. Briefly, TIGIT-huIgG1 protein was coated in 96-well plates. The HRP-linked anti-mouse IgG antibody (Cat.: 7076S, Cell Signaling Technology, USA) and substrate (Cat.: 00-4201-56, eBioscience, USA) were used to develop color absorbance signal at the wavelength of 450 nm, which was measured by using a plate reader (SpectraMax Paradigm, Molecular Devices, USA). The ELISA-positive clones were further verified by FACS using either NK92MI/huTIGIT or NK92mi/mkTIGIT cells described above. TIGIT-expressing cells (10⁵ cells/well) were incubated with ELISA-positive hybridoma supernatants, followed by binding with Alexa Fluro-647 labeled goat anti-mouse IgG antibody (Cat.: A0473, Beyotime Biotechnology, China). Cell fluorescence was quantified using a flow cytometer (Guava easyCyte 8HT, Merck-Millipore, USA).

The conditioned media from the hybridomas that showed positive signals in both ELISA and FACS screening were subjected to functional assays to identify antibodies with good functional activity in human immune cell-based assays (see following sections). The antibodies with desired functional activities were further sub-cloned and characterized.

### Subcloning and Adaptation of Hybridomas to Serum Free or Low Serum Medium

After primary screening by ELISA, FACS and functional assays as described above, the positive hybridoma clones were sub-cloned by the limiting dilution. Three positive subclones based on ELISA and FACS screening from each plate were selected and characterized by functional assays. The top antibody subclones verified through functional assays were adapted for growth in the CDM4MAb medium (Cat.: SH30801.02, Hyclone, USA) with 3% FBS.

### Expression and Purification of Monoclonal Antibodies

Hybridoma cells or 293G cells transiently transfected with an antibody expression plasmid (Cat. No. R79007, Invitrogen) was cultured either in CDM4MAb medium (Cat.: SH30801.02, Hyclone) or in Freestyle™ 293 Expression medium (Cat.: 12338018, Invitrogen), and incubated in a CO₂ incubator for 5 to 7 days at 37° C. The conditioned medium was collected through centrifugation and filtrated by passing a 0.22 μm membrane before purification. Murine or recombinant antibodies containing supernatants were applied and bound to a Protein A column (Cat.: 17127901, GE Life Sciences) following the manufacturer's guide. The procedure usually yielded antibodies at purity above 90%. The Protein A-affinity purified antibodies were either dialyzed against PBS or further purified using a HiLoad 16/60 Superdex200 column (Cat.: 17531801, GE Life Sciences) to remove aggregates. Protein concentrations were determined by measuring absorbance at 280 nm. The final antibody preparations were stored in aliquots in −80° C. freezer.

### Example 2: Cloning and Sequence Analysis of TIGIT Antibodies

Murine hybridoma clones were harvested to prepare total cellular RNAs using Ultrapure RNA kit (Cat.: 74104, QIAGEN, Germany) based on the manufacturer's protocol. The 1^{st} strand cDNAs were synthesized using a cDNA synthesis kit from Invitrogen (Cat.: 18080-051) and PCR amplification of the nucleotide sequences coding for heavy chain variable region (Vh) and kappa chain variable region (Vk) of murine mAbs was performed using a PCR kit (Cat.: CW0686, CWBio, Beijing, China). The oligo primers used for antibody cDNAs cloning of Vh and Vk were synthesized by Invitrogen (Beijing, China) based on the sequences reported previously (Brocks et al. 2001 Mol Med 7:461). PCR products were then subcloned into the pEASY-Blunt cloning vector (Cat.:C B101-02, TransGen, China) and sequenced by Genewiz (Beijing, China). The amino acid sequences of Vh and Vk regions were deduced from the DNA sequencing results.

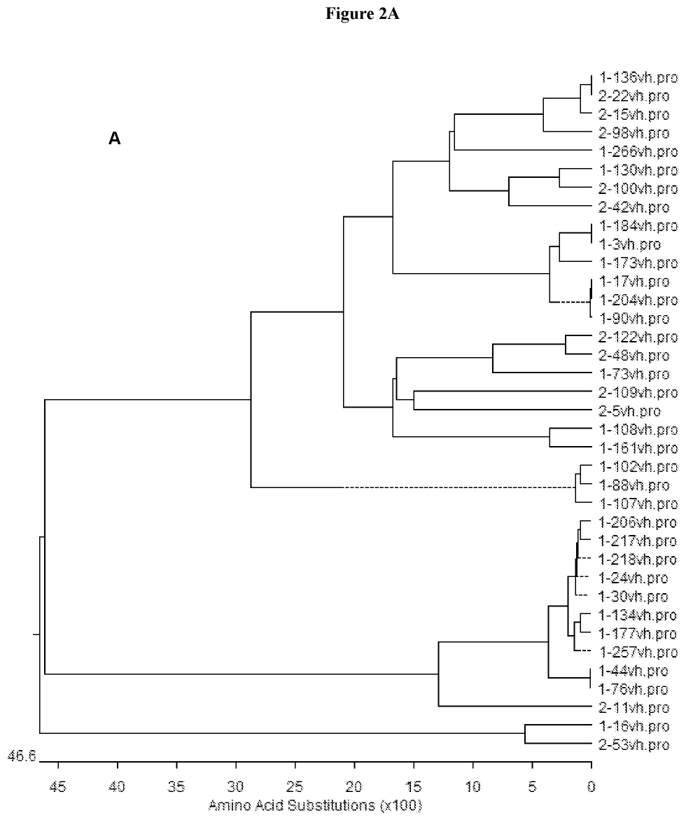

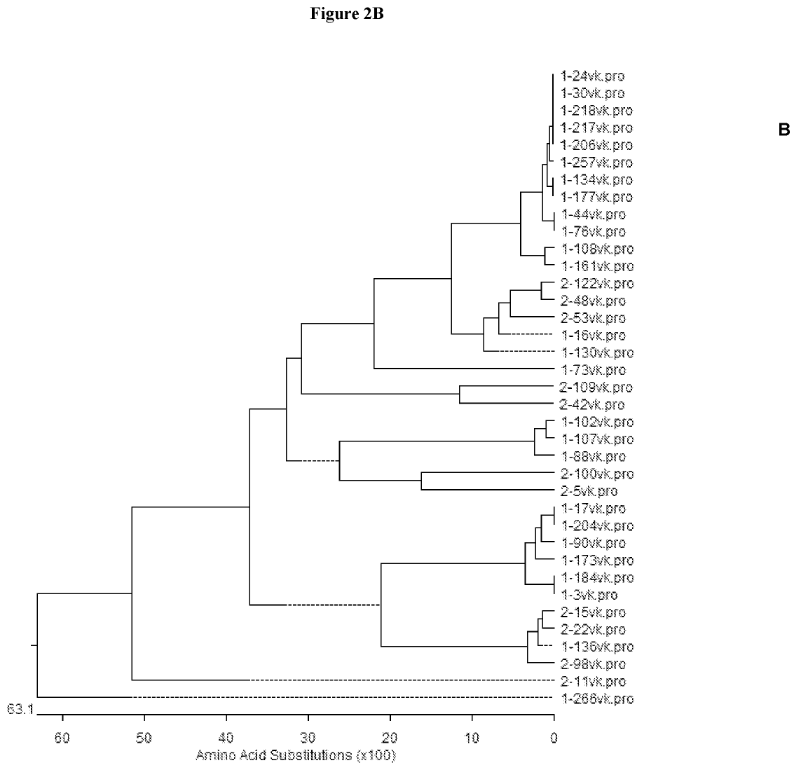

The murine mAbs were analyzed by comparing sequence homology and grouped based on sequence similarity (FIG. 2A-B). Complementary determinant regions (CDRs) were defined based on the Kabat (Wu and Kabat 1970 J. Exp. Med. 132:211-250) and IMGT (Lefranc 1999 Nucleic Acids Research 27:209-212) system by sequence annotation and by internet-based sequence analysis at the IMGT website. The amino acid sequences of a representative top clone mu1217 (Vh and Vk) were listed in Table 1 (SEQ ID NOs: 9 and 11). The CDR sequences of mu1217 were listed in Table 2 (SEQ ID NOs: 3-8).

TABLE 1
Amino acid sequences of mu1217 Vh and Vk regions

| | |
|---|---|
| mu1217 Vh | SEQ ID NO 9 |
| mu1217 Vk | SEQ ID NO 11 |

TABLE 2
CDR sequences (amino acids) of mu1217 Vh and Vk regions

| mAbs | CDR1 | CDR2 | CDR3 |
|---|---|---|---|
| mu1217, Vh | SEQ ID NO 3 | SEQ ID NO 4 | SEQ ID NO 5 |
| mu1217, Vk | SEQ ID NO 6 | SEQ ID NO 7 | SEQ ID NO 8 |

Note:
CDR sequences are defined based on Kabat system

### Example 3: Affinity Determination of Purified Murine Anti-TIGIT Antibodies by SPR

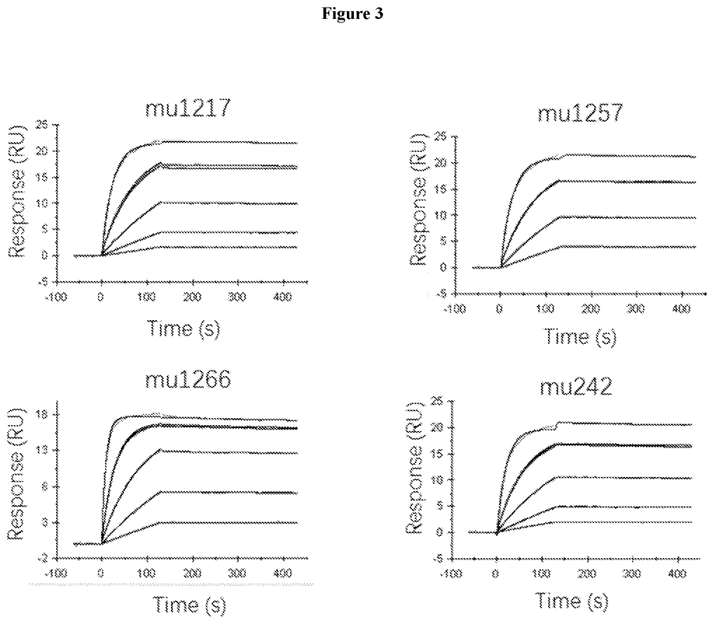

The TIGIT antibodies with high binding activities in ELISA and FACS, as well as with potent functional activities in the cell-based assays (described in Examples 1 and 2) were characterized for their binding kinetics by SPR assays using BIAcore™ T-200 (GE Life Sciences). Briefly, anti-human IgG antibody was immobilized on an activated CM5 biosensor chip (Cat. No.: BR100530, GE Life Sciences). Human Fc-tagged TIGIT was flowed over the chip surface and captured by anti-human IgG antibody. Then a serial dilution (0.12 nM to 10 nM) of purified murine antibodies were flowed over the chip surface and changes in surface plasmon resonance signals were analyzed to calculate the association rates (kₒₙ) and dissociation rates (k_{off}) by using the one-to-one Langmuir binding model (BIA Evaluation Software, GE Life Sciences). The equilibrium dissociation constant (K_{D}) was calculated as the ratio k_{off}/kₒₙ. The binding affinity profiles of top mAbs including mu1217, mu1257, mu1226 and mu242, were shown in FIG. 3 and Table 3.

TABLE 3
Binding affinities of hybridoma antibodies by SPR

| Antibodies | kₒₙ (M⁻¹s⁻¹) | k_{off} (s⁻¹) | K_{D} (nM) |
|---|---|---|---|
| mu1217 | 4.33E+06 | 3.96E−05 | 9.15E−12 |
| mu1257 | 3.99E+06 | 4.20E−05 | 1.05E−11 |
| mu1266 | 1.07E+07 | 8.49E−05 | 7.94E−12 |
| mu242 | 5.12E+06 | 7.13E−05 | 1.39E−11 |

### Example 4: Humanization of the Murine Anti-Human TIGIT mAb mu1217

### mAb Humanization and Engineering

For humanization of the mu1217, human germline IgG genes were searched for sequences that share high degrees of homology to the cDNA sequences of mu1217 variable regions by running a comparison with the human immunoglobulin gene database in IMGT. The human IGVH and IGVK genes that are present in human antibody repertoires with high frequencies (Glanville et al., PNAS 106:20216-20221 2009) and are highly homologous to mu1217 were selected as the templates for humanization.

Humanization was carried out by CDR-grafting (Methods in Molecular Biology, Vol 248: Antibody Engineering, Methods and Protocols, Humana Press) and the humanization antibodies (hu1217s) were engineered as the human IgG1mf format using an in-house developed expression vector. In the initial round of humanization, mutations from murine to human amino acid residues in framework regions were guided by the simulated 3D structure, and the murine framework residues of structural importance for maintaining the canonical structures of CDRs were retained in the 1^{st} version of humanization antibody 1217 (hu1217-1-1, with six CDRs having amino acid sequences of SEQ ID NOs: 3, 13, 5 (heavy chain CDRs) and SEQ ID NOs: 6, 7, 8 (light chain CDRs), a heavy chain variable region having an amino acid sequence of SEQ ID NO: 14 and encoded by a nucleotide sequence of SEQ ID NO: 15, and a light chain variable region having an amino acid sequence of SEQ ID No:16 and encoded by a nucleotide sequence of SEQ ID NO: 17). Specifically, CDRs of mu1217 VK (SEQ ID NO: 6-8) were grafted into the framework of human germline variable gene IGVκ3-15 with 1 murine framework residue (V₅₈) retained, resulting in the humanized Vκ sequence of Hu1217-1-1 (SEQ ID NO: 16 for amino acid sequence and SEQ ID NO: 17 for nucleotide sequence). N-terminal of H-CDR2 (SEQ ID NO: 4), H-CDR1 and H-CDR3 (SEQ ID NOs: 3 and 5) of mu1217 Vh were grafted into the framework of human germline variable gene IGVH3-7 with two murine framework (T₂₄ and I₃₇ of SEQ ID NO: 10) residues retained. In the hu1217 humanization variants, only the N-terminal half of Kabat H-CDR2 was grafted, as only the N-terminal half was predicted to be important for antigen binding according to the simulated 3D structure. The amino acid sequence and nucleotide sequence of the resultant humanized Vh sequence of Hu1217-1-1 are shown in SEQ ID NO: 14 and SEQ ID NO: 15, respectively.

Hu1217-1-1 were constructed as human full-length antibody format using in-house developed expression vectors that contain constant regions of a human IgG1 variant termed as IgG1mf (SEQ ID NO: 18) and kappa chain, respectively, with easy adapting sub-cloning sites. Expression and preparation of hu1217-1-1 antibody was achieved by co-transfection of the above two constructs into 293G cells and by purification using a protein A column (Cat.: 17543802, GE Life Sciences). The purified antibodies were concentrated to 0.5-5 mg/mL in PBS and stored in aliquots in −80° C. freezer.

Based on hu1217-1-1 template, we made several single-mutations converting the retained murine residues in framework region of Vκ to corresponding human germline residues, which include V58I in Vκ and in T24A and I37V Vh. The resulted hu1217-2A-1 (T24A), hu1217-2B-1 (I37V), and hu1217-1-2a (V58I) all had similar binding and functional activities to hu1217-1-1. All humanization mutations were made using primers containing mutations at specific positions and a site directed mutagenesis kit (Cat. No. FM111-02, TransGen, Beijing, China). The desired mutations were verified by sequencing analysis. These hu1217-derived variant antibodies were tested in binding and functional assays as described previously.

Hu1217 antibodies were further engineered by introducing mutations in CDRs and framework regions to improve molecular and biophysical properties for therapeutic use in human. The considerations include amino acid compositions, heat stability (Tₘ), surface hydrophobicity and isoelectronic points (pIs) while maintaining functional activities.

Taken together, a well-engineered version of humanized monoclonal antibody, hu1217-2-2 (SEQ ID NOs:3, 5-8, 13, and 19-21), was derived from the mutation process described as above, and characterized in detail. The results showed both hu1217-2-2 and hu1217-1-1 were very similar in binding affinity and functional activities such as inhibiting the TIGIT-mediated downstream signaling.

For affinity determination, antibodies were captured by anti-human Fc surface, and used in the affinity-assay based on surface plasmon resonance (SPR) technology. The results of SPR-determined binding profiles of anti-TIGIT antibodies were summarized in Table 4. Hu1217-2-2 and hu1217-1-1 showed very similar binding profiles with average dissociation constant at 0.415 nM and 0.266 nM, respectively, which are close to that of ch1217.

TABLE 4
Binding affinities of hu1217 antibodies by SPR

| | Test 1 | | | Test 2 | | | Mean |
|---|---|---|---|---|---|---|---|
| Antibodies | kₒₙ (M⁻¹s⁻¹) | k_{off} (s⁻¹) | K_{D} (nM) | kₒₙ (M⁻¹s⁻¹) | k_{off} (s⁻¹) | K_{D}(nM) | K_{D}(nM) |
| ch1217* | 1.56 × 10⁶ | 4.43 × 10⁻⁴ | 0.283 | — | — | — | NA** |
| hu1217-1-1 | 1.45 × 10⁶ | 4.48 × 10⁻⁴ | 0.309 | 1.33 × 10⁶ | 6.94 × 10⁻⁴ | 0.520 | 0.415 |
| hu1217-2-2 | 1.80 × 10⁵ | 2.29 × 10⁻⁴ | 0.127 | 1.50 × 10⁶ | 6.08 × 10⁻⁴ | 0.404 | 0.266 |

*ch1217 is comprised of mu1217 variable domains fused to human IgG1mf/kappa constant regions
**NA: not available.

TABLE 5
CDRs of hu1217 antibodies

| Antibodies | CDR1 | CDR2 | CDR3 |
|---|---|---|---|
| hu1217-1-1, Vh | SEQ ID NO 3 | SEQ ID NO 13 | SEQ ID NO 5 |
| hu1217-2-2, Vh | SEQ ID NO 3 | SEQ ID NO 13 | SEQ ID NO 5 |
| hu1217-1-1, Vκ | SEQ ID NO 6 | SEQ ID NO 7 | SEQ ID NO 8 |
| hu1217-2-2, Vκ | SEQ ID NO 6 | SEQ ID NO 7 | SEQ ID NO 8 |

All the humanization antibodies shown above were also confirmed for functional activities on primary human immune cells isolated from healthy donors (described in Example 7).

### Example 5: Binding Activities of Different Versions of 1217 to Native TIGIT

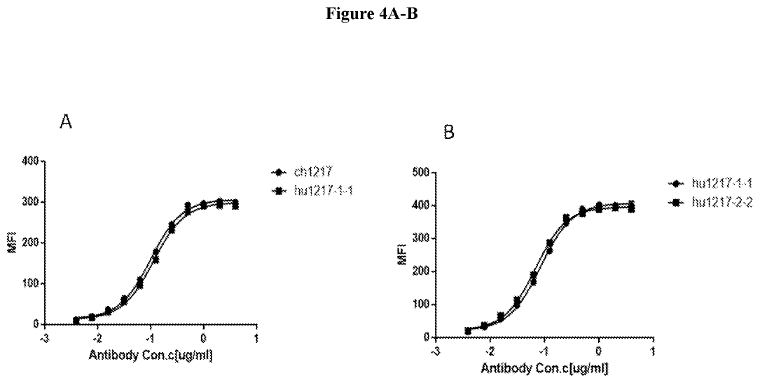

To evaluate the binding activity of anti-TIGIT antibodies to native TIGIT on living cells, NK92mi cells were engineered to over-express human TIGIT. Living NK92mi/TIGIT cells were seeded in 96-well plate and were incubated with a series of dilutions of anti-TIGIT antibodies. Goat anti-Human IgG was used as secondary antibody to detect antibody binding to the cell surface. EC₅₀ values for dose-dependent binding to human native TIGIT were determined by fitting the dose-response data to the four-parameter logistic model with GraphPad Prism. As show in FIG. 4A-B and Table 6. Both humanized 1217 antibodies, hu1217-1-1 and hu1217-2-2, showed good binding affinity to native TIGIT on living cells.

TABLE 6
EC₅₀ of dose-dependent binding of humanized
1217 variants to native TIGIT

EC₅₀(ug/mL)

| Antibodies | Test 1 | Test 2 |
|---|---|---|
| Ch1217 | 0.100 | — |
| hu1217-1-1 | 0.114 | 0.084 |
| hu1217-2-2 | | 0.068 |

### Example 6: Anti-TIGIT Antibodies Block the Interactions of TIGIT with its Ligands PVR and PVR-L2

TIGIT binds to PVR with a high affinity (Kd: ˜1 nM), which can compete against CD266-PVR interaction (Yu X, et al. 2009 Nat. Immunol, 10:48-57)

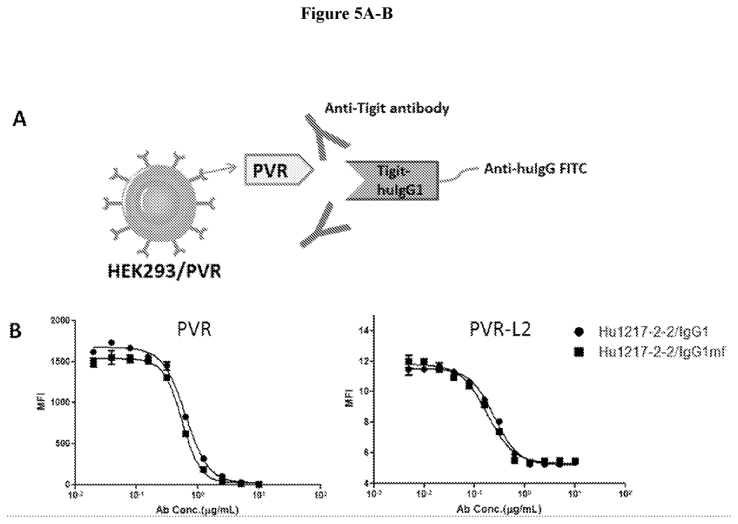

To determine whether anti-TIGIT antibodies could block TIGIT-PVR and TIGIT-PVR-L2 interactions, HEK293 cells were engineered to express high levels of PVR or PVR-L2. The resultant cell lines were named HEK293/PVR and HEK293/PVR-L2, respectively. The binding of soluble TIGIT (TIGIT-mIgG2a fusion protein) to PVR or PVR-L2 was determined by flow cytometry (FIG. 5A). The blockade of TIGIT-ligand interaction was quantitatively measured by adding serially diluted anti-TIGIT antibodies. As shown in FIG. 5B, hu1217-2-2/IgG1 (a humanized version comprising a wild-type IgG1 Fc region and having the same VH and VL sequences as hu1217-2-2/IgG1mf) and hu1217-2-2/IgG1mf could block TIGIT binding to PVR in a dose-dependent manner with IC₅₀ at 0.64 and 0.55 μg/mL, respectively. Similarly, the IC₅₀ of hu1217-2-2/IgG1 and hu1217-2-2/IgG1mf in blocking TIGIT-PVR-L2 interaction is 0.25 and 0.18 μg/mL, respectively.

### Example 7: Activation of CMV-Specific Human T Cells by Anti-TIGIT Antibodies

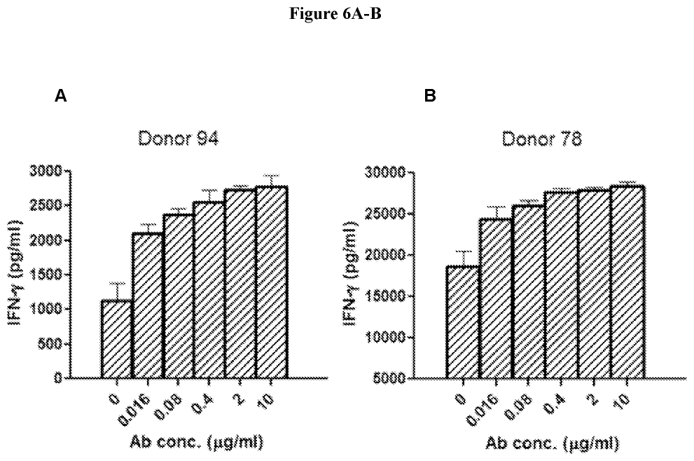

The functional activity of the TIGIT antibodies were further assessed using naturally derived T-cells that recognized human CMV PP65 peptide (NLVPMVATV, 495-503, HLA-A2.1-restricted) (Boeckh et al., 2011 J Clin Invest. 121:1673-80). Briefly, PBMCs from HLA-A2.1⁺ healthy donors were simulated with PP65 peptide (>98% purity, synthesized by GL Biochem, Shanghai) in the complete RPMI with 10% FBS for a week. The pp65-primed PBMCs were used as effector cells. Prior to assay, target cells, HCT116 cells (HLA-A2.1⁺, 10⁴), were pulsed with pp65 peptide (5 μg/mL) for 30 mins and co-cultured with equal numbers of pp65-sensitized PBMCs in 96-well plates overnight in the presence or absence of anti-TIGIT antibodies or a blank control (medium only). As shown in FIG. 6A-B, hu1217-2-2/IgG1 promoted pp65-specific T cells to secrete IFN-γ in the cell culture supernatant in a dose-dependent manner for both donors.

### Example 8: Anti-TIGIT Antibodies Enhanced NK Cell-Mediated Cytotoxicity

TIGIT is known to be constitutively expressed on natural killer (NK) cells at relatively higher levels and the interaction between TIGIT and its ligands inhibits NK cell-mediated cytotoxicity (Wang F, et al. 2015 Eur. J. Immunology 45:2886-97; Stanietsky N et al., 2009 Proc Natl Acad Sci USA 106:17858-63).

To confirm whether humanized anti-TIGIT antibodies could promote NK-mediated cytotoxicity, an NK cell line NK92MI was engineered to co-express both TIGIT and DNAM-1 receptors (NK92MI/TIGIT-DNAM-1) as an effector cell by retroviral transduction, according to the protocols described previously (Zhang et al, 2006 Cancer Res. 66: 5927-5933). A PVR-expressing lung cancer cell line SK-MES-1/PVR was established similarly as a target.

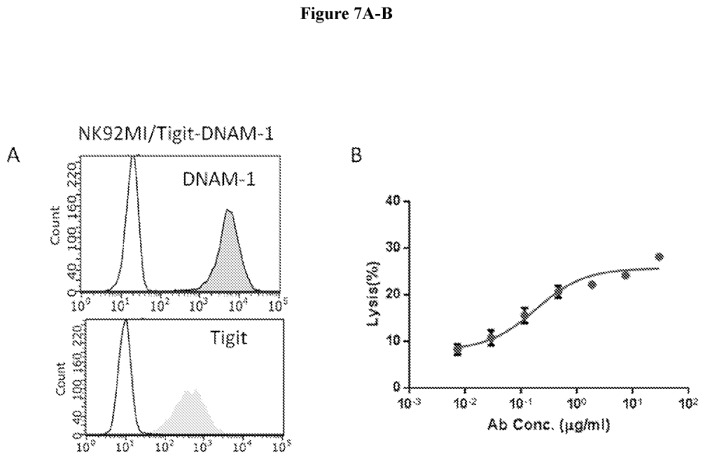

Cytotoxicity of NK92MI/TIGIT-DNAM-1 cells against SK-MES-1/PVR cells was determined by an LDH release assay using the CytoTox 96 Non-Radioactive Cytotoxicity Assay kit (Promega, Madison, WI). In brief, NK92MI/TIGIT-DNAM-1 cells (8×10⁵) were co-cultured with SK-MES-1/PVR cells (2×10⁴) in the presence of anti-TIGIT Abs (0.007-30 μg/mL) for 5 hr in 96-well V-bottom plates. LDH-release assay. Specific lysis was determined using the following equation: percentage of specific lysis=[(experimental-effector spontaneous-target spontaneous)/(target maximum−target spontaneous)]×100. The results showed that anti-TIGIT antibodies hu1217-2-2/IgG1mf enhances NK cell killing in a dose-dependent manner (EC₅₀: 0.185 μg/mL) (FIG. 7A-B).

### Example 9: Anti-TIGIT Antibodies can Reduce the Surface Expression of TIGIT Receptor Via FcγR-Mediated Trogocytosis

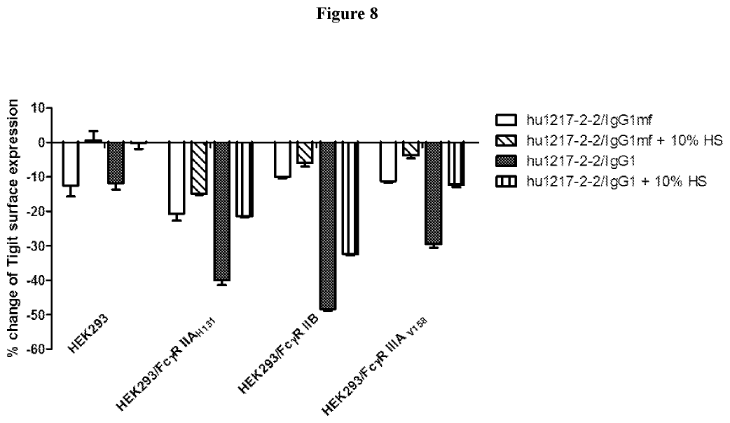

Trogocytosis is a phenomenon, in which cell surface molecules are transferred from donor cells to acceptor cells (Joly E, et al. 2003 Nat. Immunol; Machlenkin A, et al. 2008 Cancer Res.; Beum P V et al. 2008 J. Immunol; Rossi E A, et al. 2013 Blood). Antibody-induced trogocytosis via Fc γ receptors (FcγRs) leads to down-modulation of receptors on the cell surface (Carlsten et al. 2016 Clin Cancer Res; Beum et al. 2011 J. Immunology). Therefore, down-regulation of target receptor by trogocytosis may cause dampened signaling. In view of these observations, it would be possible that hu1217-2-2/IgG1 might induce trogocytosis of TIGIT receptor in the presence of FcγR⁺ cells, resulting in lower surface expression. To address this possibility, Jurkat/TIGIT cells were incubated with HEK cells expressing various FcγRs (including FcγRIIA_{H131}, FcγRIIB, FcγRIIIA_{V158}) with biotin-labeled hu1217-2-2/IgG1wt (a humanized antibody comprising the same VL and VH sequences as hu1217-2-2/IgG1mf and a wild-type IgG1 Fc region) or hu1217-2-2/IgG1mf overnight. Surface expression of TIGIT receptor was determined by with SA-APC (Biolegend). As shown in FIG. 8, hu1217-2-2/IgG1 but not hu1217-2-2/IgG1mf caused a significant reduction of TIGIT surface expression compared to the negative control human IgG-treated cells, indicating that the reduction of surface TIGIT on Jurkat/TIGIT cells are FcγR-binding dependent. In addition, presence of 10% human serum (containing high-level of endogenous IgG) could partially reduce FcγRIIA_{H131}⁻ or FcγRIIIA_{V158}_{^{−}}, but not FcγRIIB-mediated trogocytosis of TIGIT receptor, suggesting that FcγRIIB could play a critical role reducing TIGIT surface expression by anti-TIGIT mAbs (e.g., hu1217-2-2/IgG1wt) in vivo. These observations are also consistent with previous findings (Ganesan L P, et al. 2012 J Immunol 189:4981-8; Taylor R P, et al. 2015 Blood 125:762-6).

### Example 10: ADCC and CDC Effector Functions of anti-TIGIT Antibodies

The abilities of anti-TIGIT antibodies to induce ADCC and CDC in human primary PBMCs were determined using in vitro assay as described below.

### ADCC Using Human PBMCs as Target Cells

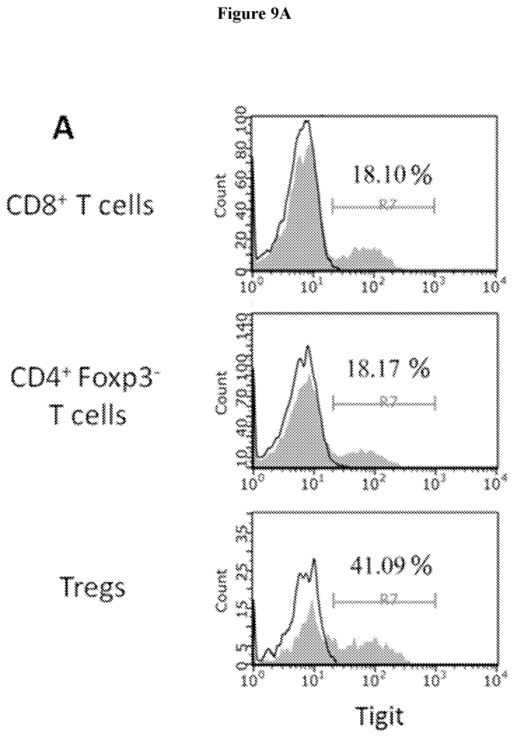

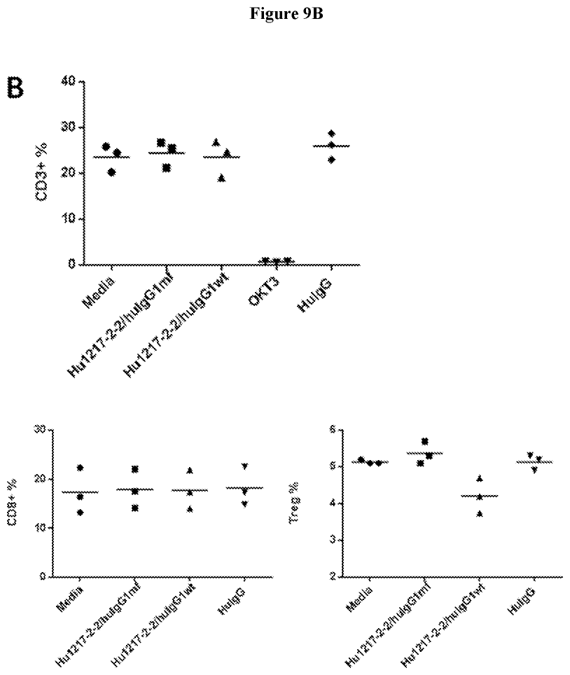

A flow cytometry-based ADCC assay was set up to determine whether TIGIT antibodies could induce ADCC in TIGIT⁺ T cells. The assay effector cell line, NK92MI/CD16V cells, was generated from NK92MI cells (ATCC) by co-transducing expression plasmids containing CD16₁₅₈ (V158 allele) and FcRγ cDNAs. Human PBMCs from healthy donors were stimulated with PHA (1 μg/ml) to up-regulate TIGIT expression. As shown in FIG. 9A, T cells, including CD4⁺ effector (CD3⁺CD4⁺Foxp3⁻), CD8⁺ and regulator T cells (CD4⁺Foxp3⁺) all expressed significant amounts of TIGIT. These activated PBMCs (from 3 healthy donors) were used as target cells. A fluorescent dye CFSE-labeled NK92MI/CD16V cells (5×10⁴) were co-cultured with equal number of target cells, for 40 hours in the presence of TIGIT antibodies (hu1217-2-2/IgG1mf or hu1217-2-2/IgG1wt, 30 μg/mL) or control antibodies (the positive control anti-CD3 antibody OKT3 (5 μg/ml, Biolegend) or a negative control human IgG, 30 μg/mL). Compared with human IgG and hu1217-2-2/IgG1mf, hu1217-2-2/IgG1wt could lead to moderate reduction of Tregs via ADCC. However, no significant ADCC effects were observed in total T cells and CD8⁺ T cells (FIG. 9B).

### CDC Using Human PBMCs as Target Cells

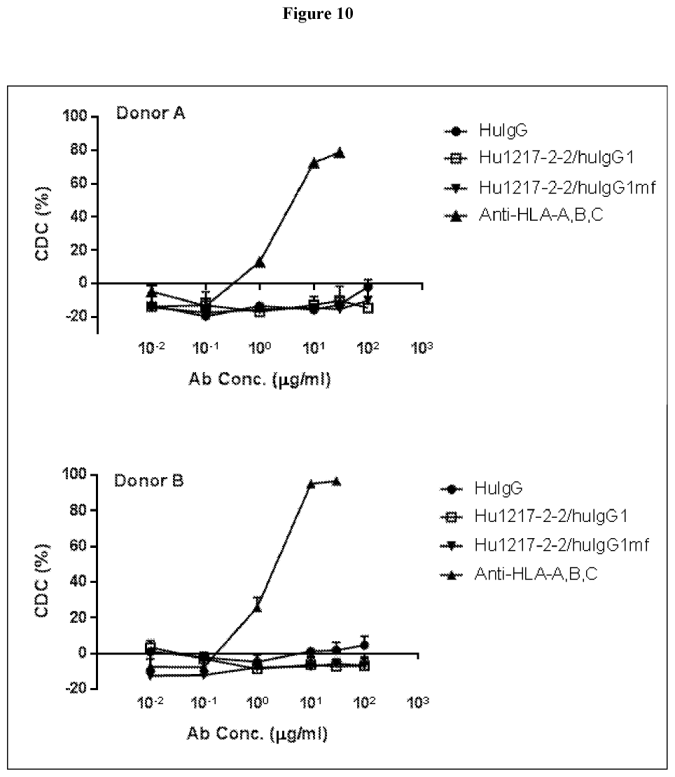

Whether hu1217-2-2/IgG1mf and hu1217-2-2/IgG1wt would trigger CDC was determined by using pre-activated human PBMCs and fresh autologous sera from healthy donors. Cell lysis by CDC was determined by a Celltiter glo assay kit (Promega, Beijing, China). In brief, PBMCs were pre-activated with PHA (10 μg/mL) for 3 days, and then were incubated in RPMI1640 plus autologous serum (15%) and anti-TIGIT or control antibodies (0.01-100 μg/mL) for overnight at 37° C. The cell death due to CDC was assayed by the decrease of ATP released from viable cells after cell lysis at the end of reaction. Anti-MHC-I A, B, C was used as a positive control. The fluorescence readout was conducted using a 96-well fluorometer (PHERA Star FS, BMG LABTECH), and the CDC activities were calculated from the relative fluorescence unit (RFU) readout as follows: % CDC activity=[(RFU test−RFU background)/(RFU at total cell lysis−RFU background)]×100. The experimental results demonstrated that both hu1217-2-2/IgG1mf and hu1217-2-2/IgG1wt had no detactable CDC with PBMCs isolated from two different donors. In contrast, the positive control antibody, anti-MHC-I, induces significant CDC activity (FIG. 10).

### Example 11: pH Dependent Binding Affinity of Hu1217-2-2/IgG1

To investigate whether pH would influence the binding property of hu1217-2-2/IgG1, target binding SPR tests were performed in running buffers at pH 7.4 and at pH 6.0 for comparison. The antibody hu1217-2-2/IgG1 was immobilized to a CM5 chip (GE). Serial dilutions of TIGIT-his were flown over the immobilized hu1217-2-2/IgG1 in running buffer HBS at pH 7.4 or pH 6.0.

As shown by the results listed in Table 7 below, hu1217-2-2/IgG1 showed higher binding affinity (KD) and stronger binding signal (Rmax) against human TIGIT at pH 6.0 (an acidic pH which is similar to the pH of tumor microenvironment) as compared to the data obtained at pH 7.4 (physiologic pH). These results indicate a potential advantage of the antibody as a therapeutic agent targeting TIGIT-positive lymphocytes in the tumor environment, since hu1217-2-2/IgG1 might more selectively target the TIGIT-positive lymphocytes in the tumor microenvironment while have lower potential toxicity associated with activation of periphery lymphocytes.

TABLE 7
Binding affinities of hu1217-2-2/IgG1
at pH 7.4 and pH 6.0 by SPR

| pH | kₒₙ (M⁻¹s⁻¹) | k_{off} (s⁻¹) | K_{D} (nM) | Rmax(RU) |
|---|---|---|---|---|
| 7.4 | 4.34E+05 | 9.53E−05 | 2.19E−10 | 21 |
| 6.0 | 2.54E+06 | 7.60E−05 | 2.99E−11 | 37 |

### Example 12: hu1217-2-2 Antibody Toxicology

Antibody hu1217-2-2 demonstrated a comparable binding affinity in TIGIT receptor occupancy assays with CD3+ splenocytes from humanized TIGIT knock-in mice compared to CD3+ human peripheral blood mononuclear cells (with EC50 of 48.8 ng/ml versus 63.2 ng/ml, respectively). In addition, hu1217-2-2 showed a significant inhibition of GL261 tumor growth in humanized TIGIT knock-in mice at a dose of ≥0.4 mg/kg via weekly intraperitoneal dosing.

The toxicity and safety profile of hu1217-2-2 was characterized in a 4-week repeated dose toxicology study in humanized TIGIT knock-in mice and a 13-week repeated dose toxicology study in cynomolgus monkeys. hu1217-2-2 was also evaluated in a 4-week repeated dose study in humanized TIGIT knock-in mice with subcutaneous MC-38 tumors. The cynomolgus monkey was considered the relevant species for toxicity studies based upon the target sequence homology and cross-species TIGIT binding activities of hu1217-2-2.

No apparent toxicity was noted in monkeys following repeated dosing at 10, 30, or 100 mg/kg of hu1217-2-2 once every 2 weeks for 13 weeks. The toxicokinetic profile in the monkey study showed that systemic exposure appeared to be dose proportional with no sex difference. No accumulation was observed over the 13-week dosing period in monkeys. No immunotoxicity was apparent as no changes in clinical pathology or histopathology were observed.

No significant increase in cytokine release was observed from an in vitro cytokine release assay following treatment of nonactivated peripheral blood mononuclear cells with hu1217-2-2 when compared to human IgG. The results indicate that hu1217-2-2 has low probability of causing acute cytokine release syndrome.

Overall, no apparent toxicity was noted in the monkey toxicity study. No unexpected tissue cross reactivity was found in human or monkey tissues. The toxicokinetic profile showed dose proportional increases in systemic exposure without apparent accumulation or sex difference. The no-observed-adverse-effect level (NOAEL) of hu1217-2-2 was 100 mg/kg in the 13-week monkey toxicity study.

### Example 13: hu1217-2-2 Clinical Pharmacology

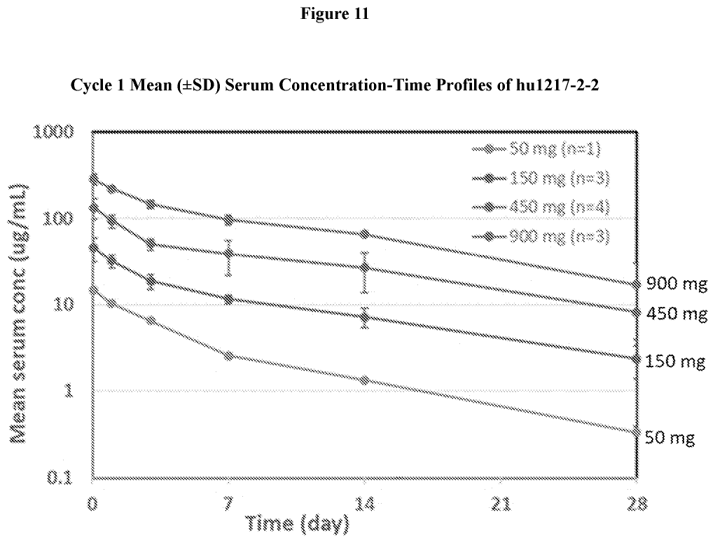

A total of 11 patients were treated with hu1217-2-2 at dose levels 50 mg (n=1), 150 mg (n=3), 450 mg (n=4), and 900 mg (n=3) in combination with BGB-A317 at a dose of 200 mg. A maximum administered dose was hu1217-2-2 at 1800 mg in combination with BGB-A317 at 200 mg Q3W. The serum concentrations of hu1217-2-2 declined in a bi-exponential manner after intravenous infusion and the hu1217-2-2 exposures (Cmax and AUC) increased approximately dose-proportionally from 50 mg to 900 mg (FIG. 11).

Peripheral TIGIT receptor occupancy data was available for the 11 patients treated with hu1217-2-2 at the doses of 50 mg (n=1), 150 mg (n=3), 450 mg (n=4), and 900 mg (n=3). Complete TIGIT receptor occupancy (100%) was observed on CD8, CD4, NK, and Treg cells in peripheral blood at all the tested dose levels, including the lowest dose of 50 mg.

### Example 14: hu1217-2-2 Alone or in Combination with BGB-A317 Promotes IFN-γ Secretion in a PBMC Assay

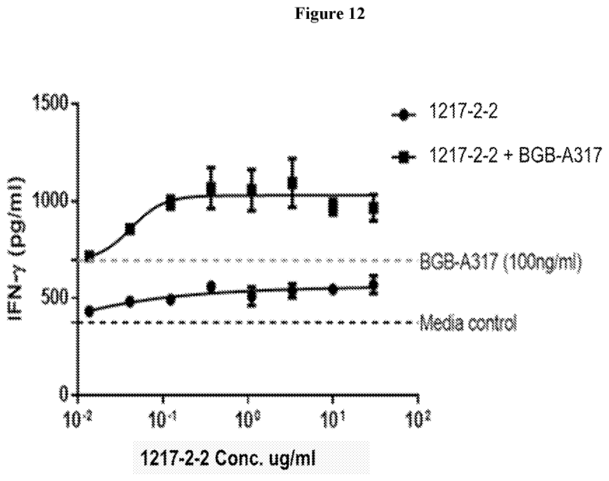

To determine whether hu1217-2-2 in combination with BGB-A317 could enhance primary human immune cell activation better than monotherapy, PBMCs from healthy donors were prestimulated to up-regulate TIGIT expression and used as effector cells. PD-L1 and T-cell engager (OS8) positive A549 cells (A549/OS8-PD-L1) were used as target cells. Pre-activated PBMCs were co-cultured with a mixture of A549/OS8-PD-L1 and A549/PD-L1 in the presence of hu1217-2-2 and BGB-A317 or either antibody alone at the indicated concentrations for 18 hours. IFN-γ production was determined by ELISA. IFN-γ secretion was used as the readout for T-cell activation. The results showed that treatment of PBMCs with hu1217-2-2 increased IFN-γ production in a dose-dependent manner. Combination of hu1217-2-2 and BGB-A317 significantly enhanced IFN-γ production relative to the increase seen for hu1217-2-2 or BGB-A317 alone, demonstrating that the combined blockade of TIGIT and PD1 can mitigate effector cell exhaustion following activation and this data is shown graphically in FIG. 12.

### Example 15: hu1217-2-2 Reduces Tumor Growth in a Mouse Glioma Tumor Model

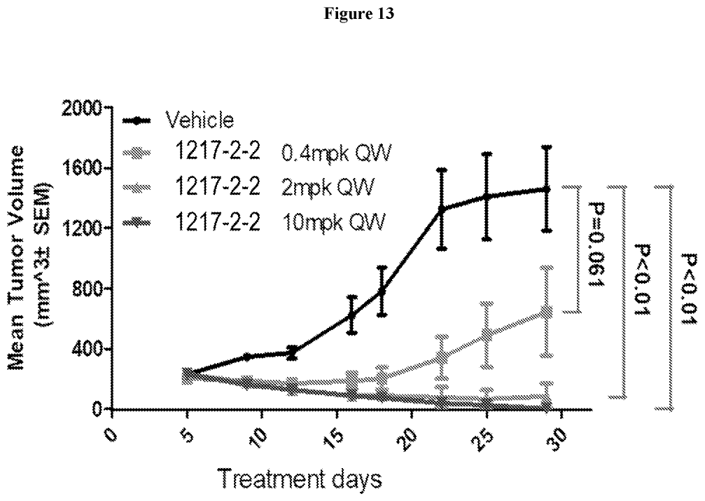

The antitumor activity of hu1217-2-2 was also tested in the GL261 mouse glioma cancer model in humanized TIGIT knock-in mice. GL261 cells (1×10⁷) were implanted subcutaneously in humanized TIGIT knock-in mice. Three days after implantation, mice were randomly allocated into 4 groups and treated with DPBS (vehicle) or hu1217-2-2 intraperitoneally for 29 days as indicated. Tumor volume was measured twice weekly. Data are presented as mean tumor volume±SEM of 12 animals in each group. As shown in FIG. 13 and Table 8, once weekly (QW) administration of hu1217-2-2 induced dose-dependent antitumor activity. On Day 29 of treatment (7 days after fourth dose), hu1217-2-2 induced significant TGI (56%, 94%, and 100% of TGI) at all doses tested (0.4, 2, and 10 mg/kg QW, respectively) (Table 8).

Blood samples (˜100 μL) were collected at predose and at 2, 8, 24, 48, 96, and 168 hours after the first dosing of hu1217-2-2 (3 mice per time point, every mouse was used for no more than 2 time points). The concentration of hu1217-2-2 in serum was determined by ELISA assay, and the serum concentration of hu1217-2-2 at each time point was determined by fitting the dose-response data to the 5-parameter logistic model. Pharmacokinetic profiles of hu1217-2-2 at the 3 doses (0.4, 2, and 10 mg/kg) were characterized for 7 days after the first injection and dose dependent exposure (Cmax 1st dose and AUC0-168h) of hu1217-2-2 was observed (Table 9). The treatment had no significant impact on animal body weight throughout the study.

TABLE 1
Dose-Dependent Efficacy of hu1217-2-2 in the GL261 Syngeneic
Tumor Model in Humanized TIGIT Knock-in Mice

| | | | | | Mean tumor | |
|---|---|---|---|---|---|---|
| | | | | | volume on | p-value vs |
| Test | Dose | | | TGI | Day 29 | vehicle |
| article | (QW) | | N | (Day 29) | (mm³) | treatment |
| Vehicle | — | | 12^{a} | — | 1459.1 | — |
| hu1217-2-2 | 0.4 | mg/kg | 12 | 56% | 644.8 | 0.0612 |
| | 2 | mg/kg | 12 | 94% | 85.0 | 0.0021 |
| | 10 | mg/kg | 12 | 100% | 1.5 | 0.0020 |

Abbreviations: QW, once weekly; TGI, tumor growth inhibition;. The p-value was calculated using t-test. TGI was calculated on Day 29 of treatment.

TABLE 2
Pharmacokinetic Parameters of hu1217-2-2 after
a Single Dose in Humanized TIGIT Knock-in Mice

| | | | C_{max, 1st dose} | AUC_{0-168 h} | t½ |
|---|---|---|---|---|---|
| Test article | Dose | | (μg/mL) | (μg/mL · h) | (day) |
| Vehicle | — | | — | — | — |
| hu1217-2-2 | 0.4 | mg/kg | 4.0 | 376.9 | 11.1 |
| | 2 | mg/kg | 16.2 | 2311.8 | 13.8 |
| | 10 | mg/kg | 98.1 | 12264.4 | 8.8 |

For Table 9, blood samples (˜100 μL) were collected at predose and at 2, 8, 24, 48, 96, and 168 hours after the first dosing of hu1217-2-2 (3 mice per time point, every mouse was used for no more than 2 time points). The concentration of hu1217-2-2 in serum was determined by ELISA assay, and the serum concentration of hu1217-2-2 at each time point was determined by fitting the dose-response data to the 5-parameter logistic model.

### Example 16: Combination of hu1217-2-2 and an Anti-PD 1 Antibody in the MC38 Mouse Colon Cancer Model in a Humanized TIGIT Knock-in Mouse Model

The antitumor activity for the combination of hu1217-2-2 with anti-mouse PD1 (muPD1) was investigated in the MC38 mouse colon cancer model in humanized TIGIT knock-in mice. MC38 tumor cells (1×10⁶) were implanted subcutaneously in humanized TIGIT knock-in mice. Seven days after implantation, the mice were randomly allocated into 4 groups and treated with vehicle (DPBS), muPD1 (1 mg/kg intraperitoneally once every 5 days (Q5D)), hu1217-2-2 (3 mg/kg intraperitoneally Q5D), or the combination, as indicated.

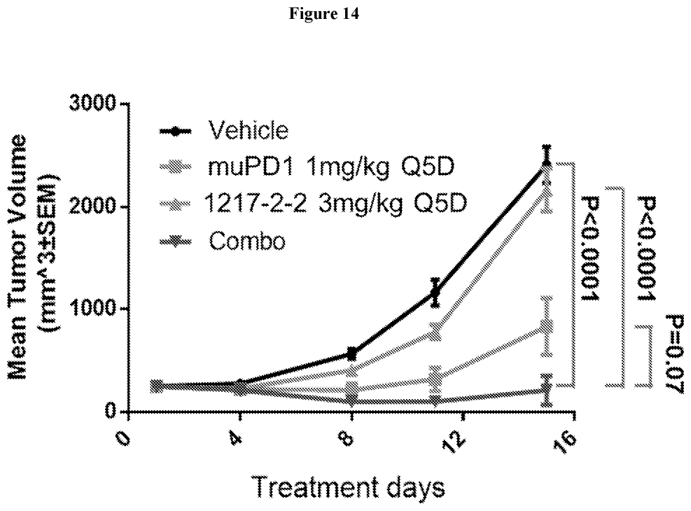

Tumor volume was measured twice weekly. Data are presented as mean tumor volume±SEM of 10 animals. The p-value was calculated using Student's t-test. The combination of hu1217-2-2 and muPD1 showed higher TGI (102%) than either antibody alone (73% for muPD1 alone or 11% for hu1217-2-2 alone) (FIG. 14 and Table 10). The treatment had no significant impact on animal body weight throughout the study.

TABLE 3
Efficacy of hu1217-2-2 in combination
with an anti-mouse PD1 antibody

| | | | | Mean tumor | |
|---|---|---|---|---|---|
| | | | | volume on | Tumor |
| Test | Dose | | TGI | Day 15 | free |
| article | (Q5D) | N | (Day 15) | (mm³) | number |
| Vehicle | — | 10 | — | 2410.1 | 0/10 |
| muPD1 | 1 mg/kg | 10 | 73% | 831.0 | 4/10 |
| hu1217-2-2 | 3 mg/kg | 10 | 11% | 2165.5 | 0/10 |
| muPD1 + | 1 mg/kg of | 10 | 102% | 208.1 | 8/10 |
| hu1217-2-2 | muPD1 + 3 mg/ | | | | |
| | kg of hu1217-2-2 | | | | |

### Example 17: Anti-TIGIT and Anti-PD1 Antibody Dosing

The doses of hu1217-2-2 ranging from 50 mg to 900 mg with a maximum of 1800 mg once every 3 weeks, in combination with 200 mg of the anti-PD1 antibody BGB-A317 once every 3 weeks were tested in an ongoing Ph1/1b study. All the tested hu1217-2-2 dose levels cleared the dose limiting toxicity (DLT) window without any significant safety or tolerability events. hu1217-2-2 exposures increased in an approximately dose-proportional manner. As such, the administered hu1217-2-2 dose of 900 mg was selected as the recommended phase II dose.

Complete TIGIT receptor occupancy was observed in circulating T cells and NK cells in peripheral blood at all the tested doses in the study. As mentioned above, data showed that 100% receptor occupancy was achieved at 50 mg. The 900 mg dose of hu1217-2-2 is expected to increase the likelihood of efficacious concentrations and saturation of TIGIT receptors in tissues completely over the entire dosing interval.

The pharmacokinetic data on hu1217-2-2 from the study indicated the lack of a significant relationship between the hu1217-2-2 exposure and patient's body weight and thus indicate that a fixed dose hu1217-2-2 would be optimal. For the combination of the hu1217-2-2 antibody with BGB-A317, the dose of hu1217-2-2 is 900 mg as a fixed dose and BGB-A317 at a fixed dose of 200 mg administered intravenously once every 3 weeks was selected. The BGB-A317 dose was selected based on comparable safety and efficacy profiles between 2 and 5 mg/kg in previous BGB-A317 studies.

### Example 18: hu1217-2-2 Antibodies in Combination with BGB-A317 Antibodies

A Phase 1 study was initiated to investigate the safety/tolerability, pharmacokinetic (PK), and preliminary antitumor activity of hu1217-2-2 in combination with BGB-A317 with or without chemotherapy in patients with unresectable locally advanced or metastatic solid tumors.

Patients enrolled in the study were treated with escalating doses of hu1217-2-2 (50, 150, 450, or 900 mg) in combination with BGB-A317 at 200 mg once every 3 weeks; all patients cleared the dose-limiting toxicity (DLT) period without toxicity that would require either removing the patient from the therapy or reducing the dose. Thus, hu1217-2-2 appears to be safe and well tolerated.

### Example 19: hu1217-2-2 Antibodies in Combination with Anti-PD1 Antibodies (BGB-A317) in the Treatment of Limited Stage Small Cell Lung Cancer

BGB-A317 as single agent or with chemotherapy was tested in a Phase 2 trial of locally advanced or metastatic nonsquamous NSCLC, squamous NSCLC, and ES-SCLC and BGB-A317 plus chemotherapy showed anticancer activity; 13 of 17 patients with small cell lung cancer (SCLC) responded to BGB-A317 plus chemotherapy in first-line treatment. Two Phase 3 studies of BGB-A317 plus platinum-doublet chemotherapy as a first-line treatment in locally advanced and metastatic nonsquamous and squamous NSCLC have demonstrated positive results of progression free survival as the primary endpoint. The hu1217-2-2 and BGB-A317 antibodies have non-overlapping anticancer mechanisms and are likely to have synergistic and/or added activity for the treatment of SCLC.

### Example 20: hu1217-2-2 Antibodies in Combination with BGB-A317 anti-PD1 Antibodies and Chemoradiotherapy in the Treatment of Limited Stage Small Cell Lung Cancer

As a novel immunotherapy combination, anti-PD-L1 plus anti-TIGIT given concurrently with radiotherapy has been evaluated in a mouse model (Grapin et al., J Immunother Cancer 2019; 7:160-71). The experiment compared the tumor response during the treatment with anti-PD-L1 plus anti-TIGIT plus radiotherapy to either single agent therapy plus radiotherapy or radiotherapy alone. In CT26 model, radiotherapy was dramatically more effective in combination with anti-TIGIT and anti-PD-L1 therapy, with a complete response rate of 9/10 (90%) mice compared to that of 2/10 (20%), 3/10 (30%), 0/10 (0%) for anti-TIGIT, anti-PD-L1 and radiotherapy alone, respectively, in 3×8 Gy hypo-fractionated radiotherapy. A complete response rate of anti-TIGIT and anti-PD-L1 in combination with 18×2 Gy normo-fractionated radiotherapy was observed in 7/12 (58.3%) mice compared to 3/12 (25%), 8/12 (66.7%), and 1/10 (10%) mice for anti-TIGIT, anti-PD-L1, and radiotherapy alone in the same fractionated RT. SCLC is characterized with relatively high TIGIT and PVR expression and high PVR expression is correlated with poor prognosis (Yu et al., Cancer Res. 2018; 1538-7445.AM2018-3637).

The novel therapeutic strategy of hu1217-2-2 and BGB-A317 combined with chemoradiotherapy seeks to augment current modest improvement in SCLC, specific to limited stage, which still only has chemotherapy in combination with radiation therapy as the standard of care. The investigation will use BGB-A317 at 200 mg intravenously once every 3 weeks combined with concurrent chemoradiotherapy for 4 cycles, followed by BGB-A317 at 200 mg intravenously once every 3 weeks.

This will be compared with BGB-A317 200 mg intravenously once every 3 weeks plus hu1217-2-2 900 mg intravenously once every 3 weeks combined with concurrent chemoradiotherapy for 4 cycles, followed by BGB-A317 200 mg intravenously once every 3 weeks plus hu1217-2-2 900 mg intravenously once every 3 weeks.

The preferred chemotherapy regimen is cisplatin 75 mg/m2 on Day 1 and etoposide 100 mg/m2 on Days 1, 2, and 3 for the first 4 cycles. Dose adjustment is allowed according to renal, hematologic, or other toxicities after the first cycle. Radiotherapy should start concurrently with chemotherapy on C1D1, with an acceptable window at the latest to coincide with Cycle 3 Day 1 with chemotherapy. The recommended total dose of 45 Gy over 3 weeks or 60-70 Gy over 6.5 weeks is at the investigator's discretion. Prophylactic Cranial Irradiation is permitted as a total dose of 25 Gy over 2 weeks (25 Gy in 10 daily fractions) per local standard of care. This regimen is detailed below in Table 11.

TABLE 11
Dose, Frequency of Administration, and Route of Administration for the
combination of hu1217-2-2 and BGB-A317 in combination with chemotherapy

| | | | | Frequency of | Route of | Duration of |
|---|---|---|---|---|---|---|
| Study Drug | Dose | | | Administration | Administration | Treatment |
| BGB-A317 | 200 mg | | | Day 1 of each cycle | Intravenous | As noted above |
| | | | | (21 days) | | |
| hu1217-2-2 | 900 mg | | | Day 1 of each cycle | Intravenous | As noted above |
| | | | | (21 days) | | |
| Cisplatin + | Cisplatin | 75 | mg/m² | Day 1 of each cycle | Intravenous | First 4 cycles |
| Etoposide | | | | (21 days) | | |
| | Etoposide | 100 | mg/m² | Days 1, 2, and 3 of | Intravenous | First 4 cycles |
| | | | | each cycle (21 days) | | |
| Carboplatin + | Carboplatin | AUC 5 | | Day 1 of each cycle | Intravenous | First 4 cycles |
| Etoposide | | | | (21 days) | | |
| | Etoposide | 100 | mg/m² | Days 1, 2, and 3 of | Intravenous | First 4 cycles |
| | | | | each cycle (21 days) | | |

Abbreviation: AUC, area under the concentration-time curve

### Example 21: hu1217-2-2 Antibodies in Combination with BGB-A317 Anti-PD1 Antibodies in the Treatment of Non-Small Cell Lung Cancer (NSCLC)

Lung cancer is the most common cancer, with approximately 2.09 million new diagnoses and 1.76 million deaths worldwide in 2018, which corresponds to the highest incidence among cancers and the most common cancer-related mortality. The disease is more common in men than women, representing 16.8% of all cancers in men and 8.8% of all cancers in women. In China, lung cancer is the leading cause of cancer-related death in both men and women, with an estimated 610,200 deaths and an estimated 733,300 new cases in the year 2015 (Chen et al., CA Cancer J Clin. 2016; 66(2):115-32). Non-small cell lung cancer (NSCLC) originates from the epithelial cells of the lung and accounts for 80% to 85% of all lung cancers. There are 3 main histological subtypes of NSCLC, adenocarcinoma which constitutes 40% of all NSCLC, squamous cell carcinoma which accounts for 25% and large cell carcinoma which constitutes 10% of all NSCLC.

The prognosis for NSCLC patients is relatively poor, although it greatly depends on the stage at which the cancer is detected. Lung cancer staging is performed worldwide according to the tumor, lymph node, and metastasis (TNM) Classification of Malignant Tumors, Seventh Edition (Goldstraw et al., J Thorac Oncol 2007; 2(8):706-14). If lung cancer is diagnosed in its earliest stages, cure is possible through surgery or chemo-radiation therapy. Unfortunately, lung cancer cases are most often detected at a relatively late stage. Approximately one third of patients with NSCLC present with locally advanced Stage III disease, including involvement of locoregional mediastinal lymph nodes or organs. The five-year survival rates for patients with Stage III disease range from 36% (Stage IIIA) to 13% (Stage IIIC). Fifty-five percent of patients with newly diagnosed NSCLC have distal metastases (Stage IV). Stage IVA patients present with contralateral lung involvement, malignant pleural effusion, and malignant pericardial effusion or with metastases in a single location outside the chest, for instance, a distal lymph node or organ such as brain, liver, or bone. Stage IVB patients present with disease that has spread to multiple locations either distal lymph nodes or organs. The 5-year survival rate for patients with Stage IV NSCLC is 5% (Siegel et al., A Cancer Journal for Clinicians 2020; 70(1):7-30). Patients with squamous NSCLC may receive platinum-based doublets with either gemcitabine, vinorelbine, or taxanes. Patients with nonsquamous NSCLC are administered pemetrexed based combination chemotherapy with carboplatin or cisplatin. Single agent pembrolizumab was approved by the US Food and Drug Administration (FDA) as first line therapy for patients with metastatic NSCLC whose tumors express a high level of PD-L1 (Tumor Proportion Score [TPS]≥50%) based on results from the KEYNOTE 024 trial (Reck et al., N Engl J Med. 2016; 375(19):1823-33). In this study, pembrolizumab showed a significant improvement in the OS rate at 6 months (80.2% versus 72.4% [95% CI: 0.4, 0.9]) and in progression-free survival (PFS; 10.3 months versus 6 months [95% CI: 6.7, NR]) compared to platinum-based chemotherapy. Pembrolizumab in combination with pemetrexed and platinum-based therapy has also been granted accelerated approval by the FDA as a first-line treatment for patients with Stage IIIB or IV nonsquamous NSCLC and no EGFR or ALK genomic aberrations based on the results from KEYNOTE 021 (Langer et al., Lancet Oncol. 2016; 17(11):1497-1508).

Upregulation of TIGIT expression in tumor-infiltrating lymphocytes has been reported in NSCLC (Tassi et al., Cancer Res. 2017; 77: 851-61). Blockade of TIGIT receptor alone or in combination with PD1/PD-L1 blockade has been shown both in vitro and in vivo to rescue functionally “exhausted” T-cells (Johnston et al., Cancer Cell. 2014; 26:923-3; Chauvin et al., J Clin Invest. 2015; 125:2046-58). In mouse models, TIGIT blockade in combination with anti-PD1/PD-L1 antibodies demonstrated significantly better antitumor efficacy than either monotherapy (Johnston et al., 2014 supra; Dixon et al., J Immunol. 2018; 200:3000-7).

This trial will evaluate the administration of hu1217-2-2 in combination with BGB-A317 in previously untreated, PD-L1-selected patients with locally advanced, unresectable, or metastatic NSCLC with no EGFR or ALK genomic aberrations. Based on the mechanism(s) of action, the combined blockade of TIGIT by hu1217-2-2 and PD1 by BGB-A317, is expected to improve efficacy than what has been seen with BGB-A317 alone. BGB-A317 200 mg was administered followed by hu1217-2-2 900 mg on Day 1 of each 21-day cycle (once every 3 weeks). An early readout of a patient treated with this dose in a case of NSCLC (squamous) showed stable disease (SD) with an −11.1 lesion change from baseline.

### Example 22: hu1217-2-2 Antibodies in Combination with BGB-A317 anti-PD1 Antibodies in the Treatment of Nasopharyngeal Carcinoma

Nasopharyngeal carcinoma (NPC) is relatively uncommon worldwide with 129,079 new cases accounting for only 0.7% of all cancers diagnosed in 2018, while >70% of new cases are in east and southeast Asia, with an age-standardized rate (world) of 3.0 per 100,000 in China. Among the endemic regions, in China, the incidence was higher in males than females with a ratio of about 2.5 to 1 in 2015 (Bray et al., CA Cancer J Clin. 2018; 68(6):394-424 and Chen et al., CA Cancer J Clin. 2016; 66(2):115-32).

The incidence and mortality rate of NPC have decreased in several endemic areas, possibly as a result of lifestyle changes, and advances in management, including the improvement of radiotherapy technology, broader application of chemotherapy, and more accurate disease staging (Lau et al., BMC Cancer 13:298, 2013; Hsu et al., Cancer Epidemiol Biomarkers Prey 15:856-861, 2006). NPC remains a leading cause of death from cancer with a global incidence of mortality of approximately 72,987 cases per year (Bray et al., supra). Depending on the degree of differentiation NPC is categorized into three pathological subtypes on the basis of World Health Organization (WHO) criteria. Differentiated tumors with surface keratin are defined as type I, whereas types II and III refer to nonkeratinizing differentiated and undifferentiated tumors, respectively. In 1991, types II and III were combined into a single category of non-keratinizing carcinoma. In regions where NPC is endemic, nonkeratinizing subtypes constitute most cases (>95%) and are invariably associated with Epstein-Barr virus (EBV) infection, whereas type I disease is more common in other parts of the world (Wei et al., Lancet 2005; 365: 2041-54; Nicholls et al., Adv Anat Path 1997; 4: 71-84). EBV infection is the most extensively studied etiological factor for NPC. On the basis of in situ hybridization techniques for EBV-encoded RNAs, the virus is detected exclusively in all tumor cells but not in normal nasopharyngeal epithelium, suggesting that EBV activation is necessary in the pathogenesis of NPC (Pathmanathan et al., N Engl J Med. 1995; 333: Chan et al., Cancer Res 2000; 60: 5365-70). NPC is endemic in Southern China and Southeast Asia. The standard first-line treatment is platinum-containing multiagent chemotherapy. However, no consensus has been reached regarding treatment beyond first-line therapy. There remains an unmet clinical need for more effective and well-tolerated novel agents.

As described earlier, anti-TIGIT antibodies provides for a potential mechanism to rescue immune cells from the immunosuppressive tumor microenvironment, thereby inducing an efficient antitumor immune response. Research showed that the TIGIT pathway cooperates with PD1 to maximize the suppression of effector tumor infiltrating lymphocytes (TILs) and promotes resistance to anti-PD1 therapy.

Standard-of-care treatment for the first line recurrent or metastatic NPC is composed of platinum containing multiagent chemotherapy; however, there are no standard treatment options beyond the first-line setting for patients with platinum refractory/recurrent or metastatic NPC. Recent clinical studies of other anti-PD1 antibodies, such as nivolumab, pembrolizumab, camrelizumab, and toripalimab monotherapies have demonstrated favorable clinical activity in patients with advanced NPC in second line or beyond treatment settings (median ORR, 20.5% to 34%; median OS, 16.5 to 17.4 months; median PFS, 1.9 to 6.5 months). Preliminary data of BGB-A317 as monotherapy in an expansion cohort of patients with NPC in a previous clinical trial is also encouraging (median ORR: 43%; median OS, not reached; median PFS, 12.4 months). But one current challenge is that PD1 inhibition benefits only a subset of refractory/recurrent or metastatic NPC patients and there remains an unmet need for more effective treatment for this patient population.

In this trial, patients will receive either BGB-A317+hu1217-2-2 or BGB-A317 alone as a comparator. Patients will receive BGB-A317 200 mg+hu1217-2-2 900 mg intravenously once every 3 weeks until disease progression as assessed by the investigator per RECIST v1.1, unacceptable toxicity, or loss of clinical benefit, whichever should occur first.

During the trial, tumor assessments will be performed every 6 weeks (±7 days) for the first 24 weeks, every 9 weeks (±7 days) for the remainder of Year 1, and every 12 weeks (±7 days) thereafter based on RECIST v1.1, regardless of dose delays to manage toxicities. To determine the PK properties of the BGB-A317 and hu1217-2-2 combination, blood samples will be collected at various timepoints. An optional blood sample will be taken at baseline (predose at Day 1 Cycle 1), at the time of first tumor response (predose at Day 1 of the following cycle) and at the end of treatment (EOT) visit after disease progression (10 mL for each timepoint).

TABLE 12
Planned Dose, Frequency of Administration, and Route
of Administration for hu1217-2-2 and BGB-A317 in NPC

| Study | | Frequency of | Route of | Duration of |
|---|---|---|---|---|
| Drugs | Dose | Administration | Administration | Treatment |
| hu1217-2-2 | 900 mg | Day 1 of each | Intravenous | as noted above |
| | | cycle (21 days) | | |
| BGB-A317 | 200 mg | Day 1 of each | Intravenous | as noted above |
| | | cycle (21 days) | | |

TABLE 13
Administration of hu1217-2-2 and BGB-A317 in NPC

| Cycle | Arm A (BGB-A317 + hu1217-2-2) | Arm B (BGB-A317) |
|---|---|---|
| Day 1, | BGB-A317 infusion over 60 (±5) | BGB-A317 infusion over |
| Cycle 1 | minutes followed by hu1217-2-2 | 60 (±5) minutes |
| and | infusion over 60 (±5) minutes | Patient monitoring |
| Cycle 2 | Patient monitoring for ≥120 minutes | for ≥60 minutes |
| Day 1, | BGB-A317 infusion over 30 (±5) | BGB-A317 infusion over |
| Cycle 3 | minutes followed by hu1217-2-2 | 30 (±5) minutes |
| onwards | infusion over 30 (±5) minutes | Patient monitoring |
| | Patient monitoring for ≥30 minutes | for ≥30 minutes |

Preliminary clinical results demonstrated that a patient with nasopharyngeal cancer did not progress after administration of hu1217-2-2 in combination with BGB-A317 after 36 weeks. The tumor lesion (TL) size was 31 mm upon initial presentation and regressed to 23 mm over the course of the 36-week treatment resulting in a 25.81% reduction in tumor size from base line and with no change from nadir.

TABLE 14

| | | Assessment | | | TL % | TL % | Derived | | | |
|---|---|---|---|---|---|---|---|---|---|---|
| | | Date | TL | TL | change | change | Target | Target | New | |
| Treatment/ | | (Study | SLD | Nadir | from | from | Lesion | Lesion | Lesion | Overall |
| Type of Disease | Visit | Day) | (mm) | (mm) | Baseline | Nadir | Response | Response | Present | Response |
| hu1217-2-2 150 mg + | Screening | (−14) | 31 | | | | | | | |
| BGB-A317 200 mg/ | 6 Weeks | (46) | 23 | 31 | −25.81 | −25.81 | SD | SD | No | SD |
| Nasopharyngeal | 12 Weeks | (87) | 25 | 23 | −19.35 | 8.70 | SD | SD | No | SD |
| Cancer | 18 Weeks | (129) | 26 | 23 | −16.13 | 13.04 | SD | SD | No | SD |
| | 24 Weeks | (172) | 25 | 23 | −19.35 | 8.70 | SD | SD | No | SD |
| | 30 Weeks | (214) | 26 | 23 | −16.13 | 13.04 | SD | SD | No | SD |
| | 36 Weeks | (256) | 23 | 23 | −25.81 | 0.00 | SD | SD | No | SD |

### Example 23: hu1217-2-2 Antibodies in Combination with BGB-A317 Anti-PD1 Antibodies in the Treatment of Esophageal Cancer

Esophageal cancer is the seventh most common cancer worldwide and the sixth most common cause of death from cancer. The highest incidence area spans from northern Iran through the Central Asian republics and into northern China. The most common histologic type of esophageal cancer is Esophageal Squamous Cell Carcinoma (ESCC), which is even more common in Eastern Europe and Asia. More than two-thirds of patients diagnosed with esophageal cancer will have advanced or metastatic disease, with a median survival of 8 to 10 months and an expected 5-year survival rate <5%. These data, combined with the relative lack of highly effective treatment, are indicative of the large unmet medical need in patients diagnosed with esophageal cancer in general and ESCC specifically.

Anti-PD1 therapies have shown superior efficacy compared with chemotherapy as the second-line treatment of ESCC. The KEYNOTE-181 trial enrolled 628 patients with recurrent locally advanced or metastatic esophageal cancer who progressed on or after one prior line of systemic treatment for advanced disease, a significant improvement was observed in the primary endpoint OS in patients treated with pembrolizumab as compared with chemotherapy (10.3 months versus 6.7 months, HR, 0.62; 95% CI: 0.46 to 0.90). The ATTRACTION-3 trial enrolled 419 patients with unresectable advanced, recurrent, or metastatic ESCC, who were refractory or intolerant to ≥1 fluoropyrimidine—and platinum-based regimen, a significant improvement was reported in the primary endpoint OS for patients treated with nivolumab as compared with the investigator's choice of taxane chemotherapy (10.9 months versus 8.4 months; HR, 0.77; 95% CI: 0.62 to 0.96). The overall survival benefit was observed regardless of PD-L1 expression level.

The administration of anti-TIGIT antibodies provides for a potential mechanism to rescue immune cells from the immunosuppressive tumor microenvironment, thereby inducing an efficient antitumor immune response. Research shows that the TIGIT pathway cooperates with PD1 to maximize the suppression of effector TILs as well as promote resistance to anti-PD1 therapy. Blocking antibodies targeting the PD1/PD-L1 pathway have achieved remarkable results in the treatment of ESCC as described in the trials above. Therefore, anti-TIGIT antibodies can significantly improve and/or extend the therapeutic benefit of anti-PD1 therapy in ESCC.

Patients with unresectable, locally advanced, recurrent or metastatic ESCC that failed from first-line chemotherapy represent a patient population with a great unmet medical need. Therefore, this trial is designed to compare the efficacy of BGB-A317 plus hu1217-2-2 versus BGB-A317 as a single agent in the treatment of patients positive for PD-L1 expression and esophageal cancer. The esophageal cancer can be unresectable, locally advanced, recurrent or metastatic ESCC.

Patients will receive BGB-A317 (200 mg) plus hu1217-2-2 (900 mg) intravenously once every 3 weeks until assessed by the investigator per RECIST v1.1, unacceptable toxicity, or patient withdrawal. Patients will receive BGB-A317 200 mg on Day 1 of each 21-day cycle (i.e., once every 3 weeks) followed by the administration of hu1217-2-2 900 mg on Day 1 of each 21-day cycle.

TABLE 15
Planned Dose, Frequency of Administration, and
Route of Administration the treatment of ESCC

| | | Timing of | Route of | Duration of |
|---|---|---|---|---|
| Study drugs | Dose | Administration | Administration | Treatment |
| hu1217-2-2 | 900 mg | Day 1 of each | Intravenous | As described |
| | | 21-day cycle | | above |
| BGB-A317 | 200 mg | Day 1 of each | Intravenous | As described |
| | | 21-day cycle | | above. |

TABLE 16
Administration of Study Drugs and Monitoring
Time for the treatment of ESCC

| Cycle | BGB-A317 Plus hu1217-2-2 |
|---|---|
| Day 1, Cycle 1 | BGB-A317 infusion over 60 (±5) minutes followed |
| and Cycle 2 | by hu1217-2-2 (Arm A) or placebo (Arm B) infusion |
| | over 60 (±5) minutes |
| | Patient monitoring for ≥120 minutes |
| Day 1, Cycle 3 | BGB-A317 infusion over 30 (±5) minutes followed |
| onwards | by hu1217-2-2 (Arm A) or placebo (Arm B) infusion |
| | over 30 (±5) minutes |
| | Patient monitoring for ≥30 minutes |

Preliminary clinical results showed that a patient with esophageal cancer did not progress after administration of hu1217-2-2 in combination with BGB-A317 after 6 weeks. The tumor lesion (TL) size was 38 mm upon initial presentation and regressed to 23 mm over the course of the treatment resulting in a 39.47% reduction from base line and nadir.

TABLE 17

| | | Assessment | | | TL % | TL % | Derived | | | |
|---|---|---|---|---|---|---|---|---|---|---|
| | | Date | TL | TL | change | change | Target | Target | New | |
| Treatment/ | | (Study | SLD | Nadir | from | from | Lesion | Lesion | Lesion | Overall |
| Type of Disease | Visit | Day) | (mm) | (mm) | Baseline | Nadir | Response | Response | Present | Response |
| hu 1217-2-2 450 mg + | Screening | (−7) | 38 | | | | | | | |
| BGB-A317 200 mg/ | 6 Weeks | (44) | 23 | 38 | −39.47 | −39.47 | PR | SD | No | SD |
| Esophageal Cancer | | | | | | | | | | |

### Example 24: hu1217-2-2 Antibodies in Combination with BGB-A317 Anti-PD1 Antibodies in the Treatment of Cervical Cancer

Cervical cancer is the fourth most common cancer and the fourth leading cause of cancer mortality in women with approximately 570,000 new diagnoses and 311,000 deaths worldwide in 2018. The estimated age-standardized incidence of cervical cancer was 13.1 per 100,000 women globally and varied widely among countries, with rates ranging from less than 2 to 75 per 100,000 women. China and India together contributed more than one-third of the global cervical cancer burden, with 106,000 cases and 48,000 deaths in China and 97,000 cases and 60,000 deaths in India (Arbyn et al., Lancet Glob Health. 2020; 8:e191-e203).

It is estimated that recurrent or metastatic cervical cancer develops in 15 to 61% of women, usually within the first 2 years after the completing primary treatment. For patients who have progressed after first-line platinum-based therapy, the treatment options are limited, and no standard of care has been established. Single cytostatic agents resulted only in limited response rates and of limited duration. This indicates significant unmet medical needs in patients with cervical cancer and/or metastatic cervical cancer.

The most significant cause of cervical cancer is persistent papillomavirus infection. Human papilloma virus (HPV) is detected in 99% of cervical tumors, particularly the oncogenic subtypes, such as HPV 16 and 18. As HPV has been recognized as the most important etiological factor in cervical cancers, therefore, cervical cancers are attractive targets for immunotherapy because viral proteins are strong immune stimulants. In June 2018, pembrolizumab received accelerated approval by the US FDA for the treatment of patients with cervical cancer with disease progression on or after chemotherapy and whose tumors express programmed death-ligand 1 (PD-L1).

Given the promising anti-tumor activity of anti-PD1 antibodies reported in this indication and given the scientific rationale that anti-TIGIT antibodies can improve the therapeutic benefit of anti-PD1 therapy, the combination of hu1217-2-2 and BGB-A317 can bring significant clinical benefit in the treatment of cervical cancer.

The trial is to evaluate the administration of hu1217-2-2 in combination with BGB-A317 in patients with cervical cancer, metastatic cervical cancer or previously treated recurrent cervical cancer. The first stage of the trial will be to administer hu1217-2-2 in combination with BGB-A317 to cervical cancer patients regardless of PD-L1 expression. The patients will be randomized at a 1:1 ratio to receive either hu1217-2-2 (900 mg every three weeks (Q3W)) in combination with BGB-A317 (200 mg Q3W) or BGB-A317 (200 mg Q3W) as monotherapy. After the first stage, a larger number of patients will be recruited. These patients will be administered hu1217-2-2 (900 mg Q3W) in combination with BGB-A317 (200 mg Q3W).

### Example 25: hu1217-2-2 Antibodies in Combination with BGB-A317 Anti-PD1 Antibodies in the Treatment of Solid Tumors

hu1217-2-2 was administered in combination with BGB-A317 in a trial directed to solid tumors. Table 18 shows the response of seven (7) patients that were administered hu1217-2-2 in combination with BGB-317, with varying doses of hu1217-2-2 while maintaining the dose of BGB-A317 at 200 mg. In the cases below, the Tumor Lesion (TL) showed regression under the combination therapy, and in six cases, resulted in stable disease (SD) with one patient having a partial response (PR). In general, the tumors regressed or did not progress after the combination treatment. For example, the gastric/gastroesophageal junction patient as the partial responder presented with a TL size of 84 mm and after 6 weeks of treatment, this was reduced to a TL size of 53 mm, resulting in a TL reduction of 36.9% from baseline and from nadir.

TABLE 18

| | | Assessment | | | TL % | TL % | Derived | | | |
|---|---|---|---|---|---|---|---|---|---|---|
| | | Date | TL | TL | change | change | Target | Target | New | |
| Treatment/ | | (Study | SLD | Nadir | from | from | Lesion | Lesion | Lesion | Overall |
| Type of Disease | Visit | Day) | (mm) | (mm) | Baseline | Nadir | Response | Response | Present | Response |
| hu1217-2-2 150 mg + | Screening | (−14) | 31 | | | | | | | |
| BGB-A317 200 mg/ | 6 Weeks | (46) | 23 | 31 | −25.81 | −25.81 | SD | SD | No | SD |
| Nasopharyngeal | 12 Weeks | (87) | 25 | 23 | −19.35 | 8.70 | SD | SD | No | SD |
| Cancer | 18 Weeks | (129) | 26 | 23 | −16.13 | 13.04 | SD | SD | No | SD |
| | 24 Weeks | (172) | 25 | 23 | −19.35 | 8.70 | SD | SD | No | SD |
| | 30 Weeks | (214) | 26 | 23 | −16.13 | 13.04 | SD | SD | No | SD |
| | 36 Weeks | (256) | 23 | 23 | −25.81 | 0.00 | SD | SD | No | SD |
| hu1217-2-2 450 mg + | Screening | (−14) | 81 | | | | | | | |
| BGB-A317 200 mg/ | 6 Weeks | (45) | 91 | 81 | 12.35 | 12.35 | SD | SD | No | SD |
| Uterine Cancer | 18 Weeks | (151) | 38 | 40 | −53.09 | −5.00 | NE | PD | No | PD |
| hu1217-2-2 450 mg + | Screening | (−7) | 84 | | | | | | | |
| BGB-A317 200 mg/ | 6 Weeks | (47) | 53 | 84 | −36.90 | −36.90 | PR | PR | No | PR |
| Gastric or | | | | | | | | | | |
| Gastroesophageal | | | | | | | | | | |
| Junction Cancer | | | | | | | | | | |
| hu1217-2-2 450 mg + | Screening | (−13) | 91 | | | | | | | |
| BGB-A317 200 mg/ | 6 Weeks | (49) | 87 | 91 | −4.40 | −4.40 | SD | SD | No | SD |
| Basal Cell Carcinoma | 12 Weeks | (85) | 90 | 87 | −1.10 | 3.45 | SD | SD | No | SD |
| | 18 Weeks | (130) | 88 | 87 | −3.30 | 1.15 | SD | SD | No | SD |
| | 24 Weeks | (175) | 89 | 87 | −2.20 | 2.30 | SD | SD | No | SD |
| | 30 Weeks | (217) | 90 | 87 | −1.10 | 3.45 | SD | SD | No | SD |
| hu1217-2-2 450 mg + | Screening | (−7) | 38 | | | | | | | |
| BGB-A317 200 mg/ | 6 Weeks | (44) | 23 | 38 | −39.47 | −39.47 | PR | SD | No | SD |
| Esophageal Cancer | | | | | | | | | | |
| hu1217-2-2 900 mg + | Screening | (−7) | 53.8 | | | | | | | |
| BGB-A317 200 mg/ | 6 Weeks | (38) | 45.7 | 53.8 | −15.06 | −15.06 | SD | SD | No | SD |
| Sarcoma | | | | | | | | | | |
| hu1217-2-2 900 mg + | Screening | (−28) | 101 | | | | | | | |
| BGB-A317 200 mg/ | 6 Weeks | (43) | 100 | 101 | −0.99 | −0.99 | SD | SD | No | SD |
| Pancreatic Cancer | | | | | | | | | | |

### Example 26: hu1217-2-2 Antibodies in Combination with BGB-A317 Anti-PD1 Antibodies in Dose Escalation

A phase 1 dose escalation study was conducted in patients with advanced, metastatic, unresectable solid tumors, for which standard therapy was ineffective, intolerable or unavailable. Patients received a dose of hu1217-2-2 at 50 mg intravenously (IV) as a single agent on cycle 1, day 1 and BGB-A317 at 200 mg IV on cycle 1, day 8. If this was tolerated, patients received four escalating doses of hu1217-2-2 ranging in dose from 150-900 mg, plus BGB-A317 200 mg sequentially on day 29 and every three weeks (Q3W) thereafter until discontinuation. A patient with nasopharyngeal cancer had stable cancer at a dose of hu1217-2-2 at 150 mg (Q3W) in combination with BGB-A317 200 mg (Q3W) for 54 weeks, the longest duration tested. At the dose of hu1217-2-2 at 450 mg (Q3W) in combination with BGB-A317 at 200 mg (Q3W), there was stable cancer in a patient with uterine cancer and another with basal cell carcinoma. At this same dose, a patient with gastric cancer had a partial response. Finally, the dose of hu1217-2-2 at 900 mg (Q3W) in combination with BGB-A317 at 200 mg (Q3W) resulted in six patients with stable cancer, 2 with renal cancer, 1 melanoma, 1 sarcoma, 1 pancreatic and 1 patient with salivary gland cancer. This dosage administration also had one partial response in a patient with mesothelioma.

### Example 27: hu1217-2-2 Antibodies in Combination with BGB-A317 and BAT1706 Antibodies for the Treatment of Hepatocellular Cancer

BAT1706 is a similar biological product of the bevacizumab injection (Bevacizumab Injection, brand name Avastin®). Bevacizumab is a recombinant humanized immunoglobulin G1 (IgG1) monoclonal antibody that binds with high affinity to human vascular endothelial growth factor (VEGF). Bevacizumab is able to selectively bind with high affinity to all human VEGF A isoforms and blocks the binding of VEGF to VEGF receptor-1 (VEGFR-1) and VEGF receptor 2 (VEGFR-2), thereby neutralizing the bioactivity of VEGF. It is currently the only antitumor angiogenesis drug that has a proven clinical efficacy in extending the overall survival and progression-free survival in patients with cancer. Several randomized, controlled Phase II and III studies have shown that treatment with bevacizumab in combination with standard chemotherapy resulted in a statistically significant improvement in overall survival, progression-free survival, and overall remission rates compared with the use of standard chemotherapy alone.

The results of comparative in vitro pharmacodynamic research showed that BAT1706 and bevacizumab both exhibited specific binding with VEGF consistent with literature reports. The biological activity of BAT1706 was measured using human umbilical vein endothelial cells (HUVEC) proliferation inhibition testing. The results demonstrated that BAT1706 exhibited a similar dose-dependent relationship with the growth of HUVEC to bevacizumab, with both exhibiting potent neutralizing efficacy against VEGF. The biological activity of BAT1706 was found to be comparable to that of bevacizumab within the predetermined range of (1.0±0.2)×104 U/mg.

The anti-tumorigenic effect of BAT1706 and bevacizumab in several models of human cancer (NSCLC, ovarian cancer, and rhabdomyosarcoma) was compared. NCI-H358 human NSCLC cells were subcutaneously inoculated in non-obese diabetic/severe combined immunodeficiency (NOD/SCID) female mice to establish a human NSCLC xenograft model. Animals were divided into the following 5 groups with each group consisting of 8 female mice:

Vehicle control group

bevacizumab 5.0 mg/kg group
bevacizumab 0.5 mg/kg group
BAT1706 5.0 mg/kg group
BAT1706 0.5 mg/kg group.

The tumors were allowed to grow to a mean volume of 128 mm³. Thereafter, treatments were administered by tail vein injection on Days 1, 3, and 5 of each week for the subsequent 3 weeks. Efficacy was measured by tumor weight, tumor volume, and relative tumor growth inhibition (TGI). Tumor latency was also observed, but not assessed for statistical significance. In this study, the target tumor weight used to assess tumor latency compared with the vehicle control group was the same (600 mm3) for BAT1706 and bevacizumab. The mean tumor volume of BAT1706 5 mg/kg and bevacizumab 5 mg/kg groups, but not the BAT1706 0.5 mg/kg and bevacizumab 0.5 mg/kg groups, demonstrated a statistically significant difference from the vehicle control group 1 day after dose termination. The tumor weight data were consistent with the tumor volume data. Notably, the tumor volume and tumor weight results for the paired doses (0.5 or 5 mg/kg) of BAT1706 and bevacizumab were not statistically different. The relative TGI (%) of BAT1706 5 mg/kg and bevacizumab 5 mg/kg was 94% and 93%, respectively. The relative TGI(%) of BAT1706 at 0.5 mg/kg and bevacizumab at 0.5 mg/kg was 67% and 63%, respectively. In sum, BAT1706 showed similar efficacy as bevacizumab in a dose-dependent manner.

A similar result was seen in an SK-OV-3 human ovarian cancer cell xenograft model mouse model and a 673 human rhabdomyosarcoma cell xenograft mouse model wherein BAT1706 showed similar efficacy as bevacizumab in a dose-dependent manner. All treatment groups in both studies showed no deaths or signs of serious toxicity and both antibodies were well tolerated throughout the treatment.

A Phase I study conducted in New Zealand assessed the safety of BAT1706 and compared between EU-bevacizumab and US-bevacizumab in healthy subjects. A total of 125 healthy subjects received a single, 1 mg/kg administration of 1 of 3 study drugs: BAT1706, EU-bevacizumab, or US-bevacizumab, as a 90- minute IV infusion and the study results revealed that a single IV infusion of BAT1706 is safe and well tolerated, associated with only mild injection site reactions.

A separate Phase I study conducted in China (BAT1706-002-CR) also assessed the safety of BAT1706 and compared with bevacizumab (European) in healthy subjects. A total of 80 healthy subjects received a single, 1 mg/kg administration of study drug (39 subjects in BAT1706 group and 41 subjects in bevacizumab group). The study results revealed that both the drugs showed good safety and tolerance characteristics. No anti-drug antibody positive result was reported for any subject in either the New Zealand or China study.

Hepatocellular carcinoma (HCC) is a major global health problem, accounting for 85-90% of all reported cases of liver cancer (a term with which HCC is often used interchangeably) (El-Serag et al., Gastroenterology 2007; 132(7):2557-76). According to the World Health Organization's GLOBOCAN 2012 database, liver cancer was the sixth most common type of cancer that year, with 782,000 new cases worldwide; it was also the second most common cause of cancer-related mortality, responsible for an estimated 746,000 deaths (Torre et al., CA Cancer J Clin. 2015; 65(2):87-108). Most HCC cases (>80%) occur in Eastern Asia and in sub-Saharan Africa, with typical incidence rates of >20 per 100,000 individuals. China alone accounts for approximately 50% of both new HCC cases and HCC-related deaths worldwide (Torre, supra). Southern European countries, such as Spain, Italy, and Greece, tend to have more moderate incidence rates (approximately 10 to 20 per 100,000 individuals), whereas North America, South America, Northern Europe, and Oceania have a relatively low incidence of HCC (<5 per 100,000 individuals) (El-Serag et al., Gastroenterology. 2012; 142(6):1264-73).

As described earlier, anti-PD-1/PD-L1 inhibitor monotherapy has shown clinical benefit in previously treated HCC (Qin et al., Lancet Oncol. 2020; 21(4):571-80). However, it does not show significant improvement compared with sorafenib in first-line HCC patients (Yau et al., Ann Oncol. 2019; 30, (Suppl 5); v874-v75). Given the promising anti-tumor activity of anti-PD-1 antibodies reported in HCC and given that TIGIT may improve the therapeutic benefit of anti-PD-1 therapy, the combination of hu1217-2-2 and BGB-A317 can bring about significant clinical benefit in this indication.

Single-agent activity has been observed with bevacizumab in HCC patients (Boige et al., Oncologist. 2012; 17:1063-72; Siegel J Clin Oncol 2008; 26: 2992-8). In combination with PD-L1 inhibitors, bevacizumab has shown immunomodulatory effects in other tumor types, with promising clinical benefits and a good safety profile (Wallin et al., Nat Commun. 2016; 7:12624). The positive results of the IMbrave150 study and Orient32 study demonstrate the synergistic benefit of combining anti-PD-1/PD-L1 inhibitors with antiangiogenic agents.

In a study to investigate the efficacy and safety of hu1217-2-2 in combination with BGB-A317 plus BAT1706, as a first-line treatment in patients with advanced HCC, the study will enroll approximately 90 patients randomized in a 2:1 ratio to one of 2 treatment arms:
Arm A (n=60): hu1217-2-2 900 mg intravenously once every 3 weeks (dosed in 21-day cycles)+BGB-A317 200 mg intravenously once every 3 weeks (dosed in 21-day cycles)+BAT1706 15 mg/kg intravenously once every 3 weeks (dosed in 21-day cycles).
Arm B (n=30): BGB-A317 200 mg intravenously once every 3 weeks (dosed in 21-day cycles)+BAT1706 15 mg/kg intravenously once every 3 weeks (dosed in 21-day cycles).

As disclosed above, targeting TIGIT provides a potential mechanism to rescue immune cells from the immunosuppressive tumor microenvironment, thereby inducing an efficient antitumor immune response. Research shows that the TIGIT pathway cooperates with PD-1 to maximize the suppression of effector tumor infiltrating lymphocytes (TILs) as well as promote resistance to anti-PD-1 therapy. Antibodies targeting the PD-1/PD-L1 pathway have achieved results in the treatment of HCC, therefore, treatment with hu1217-2-2 in combination with BGB-A317 and BAT1706 can significantly improve the treatment of HCC.

1. A method of cancer treatment, the method comprising administering to a subject an effective amount of an anti-TIGIT antibody or antigen-binding fragment thereof in combination with an anti-PD1 antibody or antigen binding fragment thereof.

2. The method of claim 1, wherein the method comprises administering to a subject an effective amount of an antibody or antigen-binding fragment thereof, which specifically binds to human TIGIT and comprises:
(i) a heavy chain variable region that comprises (a) a HCDR (Heavy Chain Complementarity Determining Region) 1 of SEQ ID NO: 3, (b) a HCDR2 of SEQ ID NO: 13, and (c) a HCDR3 of SEQ ID NO:5; and a light chain variable region that comprises (d) a LCDR (Light Chain Complementarity Determining Region) 1 of SEQ ID NO:6, (e) a LCDR2 of SEQ ID NO:7, and (f) a LCDR3 of SEQ ID NO:8; or
(ii) a heavy chain variable region that comprises (a) a HCDR1 of SEQ ID NO:3, (b) a HCDR2 of SEQ ID NO:4, and (c) a HCDR3 of SEQ ID NO:5; and a light chain variable region that comprises: (d) a LCDR1 of SEQ ID NO:6, (e) a LCDR2 of SEQ ID NO:7, and (f) a LCDR3 of SEQ ID NO: 8;
in combination with an anti-PD1 antibody.

3. The method of claim 2, wherein the anti-TIGIT antibody or antigen-binding fragment thereof comprises:
(i) a heavy chain variable region (VH) that comprises SEQ ID NO:19, and a light chain variable region (VL) that comprises SEQ ID NO: 21;
(ii) a heavy chain variable region (VH) that comprises SEQ ID NO: 14, and a light chain variable region (VL) that comprises SEQ ID NO: 16; or
(iii) a heavy chain variable region (VH) that comprises SEQ ID NO: 9, and a light chain variable region (VL) that comprises SEQ ID NO: 11.

4. The method of claim 1, wherein the anti-PD1 antibody comprises an antibody or an antigen binding fragment thereof which specifically binds human PD1, and comprises: a heavy chain variable region that comprises (a) a HCDR1 of SEQ ID NO: 25, (b) HCDR2 of SEQ ID NO: 26, and (c) HCDR3 of SEQ ID NO: 27; and a light chain variable region that comprises (d) LCDR1 of SEQ ID NO: 28, (e) LCDR2 of SEQ ID NO: 29, and (f) LCDR3 of SEQ ID NO: 30.

5. The method of claim 4, wherein the anti-PD1 antibody or antigen binding fragment thereof which specifically binds human PD1 and comprises a heavy chain variable region (VH) comprising an amino acid sequence of SEQ ID NO:32 and a light chain variable region (VL) comprising an amino acid sequence of SEQ ID NO: 34.

6. The method of claim 4 wherein the anti-PD1 antibody comprises an IgG4 constant domain comprising SEQ ID NO: 35.

7. The method of claim 1, wherein the anti-TIGIT antibody is an antibody fragment selected from the group consisting of Fab, Fab′-SH, Fv, scFv, and (Fab′)2 fragments.

8. The method of claim 1, wherein the anti-PD1 antibody is an antibody fragment selected from the group consisting of Fab, Fab′-SH, Fv, scFv, and (Fab′)2 fragments.

9. The method of claim 1, further comprising administering an effective amount of an anti-VEGF antibody.

10. The method of claim 9, wherein the anti-VEGF antibody is bevacizumab or BAT1706.

11. The method of claim 1, wherein the cancer is selected from the group consisting of breast cancer, colon cancer, pancreatic cancer, head and neck cancer, gastric cancer, kidney cancer, liver cancer, small cell lung cancer, non-small cell lung cancer, esophageal cancer, ovarian cancer, uterine cancer, cervical cancer, skin cancer, mesothelioma, lymphoma, leukemia, myeloma or sarcoma.

12. The method of claim 9, wherein:
(a) the small cell lung cancer is limited stage small cell lung cancer;
(b) the head and neck cancer is nasopharyngeal cancer;
(c) the esophageal cancer is esophageal squamous cell carcinoma;
(d) the gastric cancer is gastric or gastroesophageal junction cancer;
(e) the cervical cancer is recurring or metastatic cervical cancer;
(f) the skin cancer is basal cell carcinoma; and/or (g) the kidney cancer is hepatocellular carcinoma.

13. The method of claim 9, wherein the cancer is non-small cell lung cancer.

14.-15. (canceled)

16. The method of claim 9, wherein the cancer is uterine cancer.

17.-19. (canceled)

20. The method of claim 9, wherein the cancer is pancreatic cancer.

21. (canceled)

22. The method of claim 1, further comprising the administration of chemotherapy.

23. The method of claim 22, wherein the chemotherapy is chemoradiotherapy.

24. The method of claim 1, wherein the anti-PD1 antibody is dosed at 200 mg every three weeks.

25. The method of claim 24, wherein the anti-TIGIT antibody is dosed at a range of 50 mg-900 mg.

26. The method of claim 25, wherein the anti-TIGIT antibody is dosed at 50 mg, 150 mg, 450 mg, or 900 mg every three weeks.

27.-29. (canceled)


SEQUENCE LISTING (35 sequences)

SEQ ID NO: 1 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 2 - protein, 120 aa, Homo sapiens
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIP

SEQ ID NO: 3 - protein, 5 aa, Mus musculus
DYYMY

SEQ ID NO: 4 - protein, 17 aa, Mus musculus
YITKGGGSTYYPDTVKG

SEQ ID NO: 5 - protein, 10 aa, Mus musculus
QTNYDFTMDY

SEQ ID NO: 6 - protein, 11 aa, Mus musculus
KASQDVGTSVA

SEQ ID NO: 7 - protein, 7 aa, Mus musculus
WASARHT

SEQ ID NO: 8 - protein, 9 aa, Mus musculus
QQYSSYPLT

SEQ ID NO: 9 - protein, 119 aa, Mus musculus
EVKLVESGGGLVQPGGSLKLSCATSGFTFSDYYMYWIRQTPEKRLEWVAYITKGGGSTYYPDTVKGRFTISRDNAKNTLYLQVSRLKSEDTAIYYCARQTNYDFTMDYWGQGTSVTVSS

SEQ ID NO: 10 - DNA, 357 bp, Mus musculus
gaagtgaagctggtggagtctgggggaggcttagtgcagcctggagggtccctgaaactctcctgtgcaacctctggattcactttcagtgactattacatgtattggattcggcagactccagagaagaggctggagtgggtcgcatacattactaagggtggtggtagcacctattatccagacactgtaaagggccgattcaccatctccagagacaatgccaagaacaccctgtacctgcaagtgagccgtctgaagtctgaggacacagccatatattactgtgcaagacagactaactacgactttactatggactactggggtcaaggaacctcagtcacggtctcctca

SEQ ID NO: 11 - protein, 107 aa, Mus musculus
DIVMTQSHKFMSTSVGDRVSIICKASQDVGTSVAWYQQKPGQSPNLLIYWASARHTGVPDRFTGSGSGTDFTLTISNVQSEDLADYFCQQYSSYPLTFGAGTKLELK

SEQ ID NO: 12 - DNA, 321 bp, Mus musculus
gacattgtgatgacccagtctcacaaattcatgtccacatcagtaggagacagggtcagcatcatctgcaaggccagtcaggatgtgggtacttctgtagcctggtatcaacagaaaccagggcaatctcctaacctactgatttactgggcatccgcccggcacactggagtccctgatcgcttcacaggcagtggatctgggacagatttcactctcaccattagcaatgtacagtctgaagacttggcagattatttctgtcagcaatatagcagttatcctctcacgttcggtgctgggaccaagctggagctgaaa

SEQ ID NO: 13 - protein, 17 aa, Artificial Sequence
  hu1217-1-1 HCDR2
YITKGGGSTYYPDSVKG

SEQ ID NO: 14 - protein, 119 aa, Artificial Sequence
  hu1217-1-1 VH pro
EVQLVESGGGLVQPGGSLRLSCATSGFTFSDYYMYWIRQAPGKGLEWVAYITKGGGSTYYPDSVKGRFTISRDNAKNTLYLQMNSLRAEDTAVYYCARQTNYDFTMDYWGQGTLVTVSS

SEQ ID NO: 15 - DNA, 357 bp, Artificial Sequence
  hu1217-1-1 VH DNA
gaggtgcagctggtggagagcggaggaggactggtgcagcctggaggcagcctgagactgagctgcgccaccagcggcttcaccttctccgactactacatgtactggatcaggcaggcccctggcaaaggcctggagtgggtggcctacatcaccaagggcggcggcagcacctactaccccgatagcgtgaagggcaggttcaccatcagcagggacaacgccaagaacaccctgtacctgcagatgaacagcctgagggccgaggataccgccgtgtactactgcgccaggcagaccaactacgacttcaccatggactactggggccagggcacactggtgaccgtgagcagc

SEQ ID NO: 16 - protein, 107 aa, Artificial Sequence
  hu1217-1-1 VK pro
EIVMTQSPATLSVSPGERATLSCKASQDVGTSVAWYQQKPGQAPRLLIYWASARHTGVPARFSGSGSGTEFTLTISSLQSEDFAVYYCQQYSSYPLTFGGGTKVEIK

SEQ ID NO: 17 - DNA, 321 bp, Artificial Sequence
  hu1217-1-1 VK DNA
gagatcgtgatgacccagagccctgccacactgagcgtgagccctggcgagagagccaccctgagctgcaaggccagccaggatgtgggcaccagcgtggcctggtaccagcagaaacccggccaggctcccaggctgctgatctactgggccagcgccagacacacaggcgtgcctgccagatttagcggcagcggcagcggcaccgagtttaccctgaccatcagcagcctgcagtccgaggacttcgccgtgtactactgccagcagtacagcagctaccccctgacattcggcggcggcaccaaggtggagatcaag

SEQ ID NO: 18 - protein, 329 aa, Artificial Sequence
  IgG1mf
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 119 aa, Artificial Sequence
  hu1217-2-2 VH pro
EVQLVESGGGLVQPGGSLRLSCAASGFTFSDYYMYWVRQAPGKGLEWVAYITKGGGSTYYPDSVKGRFTISRDNAKNTLYLQMNSLRAEDTAVYYCARQTNYDFTMDYWGQGTLVTVSS

SEQ ID NO: 20 - DNA, 357 bp, Artificial Sequence
  hu1217-2-2 VH DNA
gaggtgcagctggtggagagcggaggaggactggtgcagcctggaggcagcctgagactgagctgcgccgccagcggcttcaccttctccgactactacatgtactgggtcaggcaggcccctggcaaaggcctggagtgggtggcctacatcaccaagggcggcggcagcacctactaccccgatagcgtgaagggcaggttcaccatcagcagggacaacgccaagaacaccctgtacctgcagatgaacagcctgagggccgaggataccgccgtgtactactgcgccaggcagaccaactacgacttcaccatggactactggggccagggcacactggtgaccgtgagcagc

SEQ ID NO: 21 - protein, 107 aa, Artificial Sequence
  hu1217-2-2 VK pro
EIVMTQSPATLSVSPGERATLSCKASQDVGTSVAWYQQKPGQAPRLLIYWASARHTGIPARFSGSGSGTEFTLTISSLQSEDFAVYYCQQYSSYPLTFGGGTKVEIK

SEQ ID NO: 22 - DNA, 321 bp, Artificial Sequence
  hu1217-2-2 VK DNA
gagatcgtgatgacccagagccctgccacactgagcgtgagccctggcgagagagccaccctgagctgcaaggccagccaggatgtgggcaccagcgtggcctggtaccagcagaaacccggccaggctcccaggctgctgatctactgggccagcgccagacacacaggcatccctgccagatttagcggcagcggcagcggcaccgagtttaccctgaccatcagcagcctgcagtccgaggacttcgccgtgtactactgccagcagtacagcagctaccccctgacattcggcggcggcaccaaggtggagatcaag

SEQ ID NO: 23 - protein, 119 aa, Artificial Sequence
  10A7 VH pro
EVQLVESGGGLVKPGGSLKLSCAASGFTFSSFTMHWVRQAPEKGLEWVAYIRSGSGIVFYADTVRGRFTISRDNAKNTLFLQMTSLRSEDTAMYYCARRPLGHNTFDSWGQGTLVTVSS

SEQ ID NO: 24 - protein, 113 aa, Artificial Sequence
  10A7 VK pro
DIVMSQSPSSLAVSAGEKVTMSCKSSQSLYYSGVKENLLAWYQQKPGQSPKLLIYYASIRFSGVPDRFTGSGSGTDFTLTISSVQAEDLAVYYCQQGINNPLTFGGGTKLEIK

SEQ ID NO: 25 - protein, 10 aa, Artificial Sequence
  BGB-A317 HCDR1
GFSLTSYGVH

SEQ ID NO: 26 - protein, 16 aa, Artificial Sequence
  BGB-A317 HCDR2
VIYADGSTNYNPSLKS

SEQ ID NO: 27 - protein, 12 aa, Artificial Sequence
  BGB-A317 HCDR3
ARAYGNYWYIDV

SEQ ID NO: 28 - protein, 11 aa, Artificial Sequence
  BGB-A317 LCDR1
KSSESVSNDVA

SEQ ID NO: 29 - protein, 7 aa, Artificial Sequence
  BGB-A317 LCDR2
YAFHRFT

SEQ ID NO: 30 - protein, 9 aa, Artificial Sequence
  BGB-A317 LCDR3
HQAYSSPYT

SEQ ID NO: 31 - DNA, 354 bp, Artificial Sequence
  4B6 DNA-VH
caggtgcagctgcaggagtcgggaccaggactggtgaagccttcggagaccctgtccctcacctgcactgtctctgggttttcattaaccagctatggtgtacactggatccggcagcccccagggaagggactggagtggatcggggtcatatacgccgatggaagcacaaattataatccctccctcaagagtcgagtgaccatatcaaaagacacctccaagaaccaggtttccctgaagctgagctctgtgaccgctgcggacacggccgtgtattactgtgcgagagcctatggtaactactggtacatcgatgtctggggccaagggaccacggtcaccgtctcctca

SEQ ID NO: 32 - protein, 118 aa, Artificial Sequence
  4B6 VH
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 33 - DNA, 321 bp, Artificial Sequence
  4B6 VL
gacatcgtgatgacccagtctccagactccctggctgtgtctctgggcgagagggccaccatcaactgcaagtccagcgagagtgtgagtaatgatgtagcttggtaccagcagaaaccaggacagcctcctaagctgctcattaactatgcatttcatcgcttcactggggtccctgaccgattcagtggcagcgggtatgggacagatttcactctcaccatcagcagcctgcaggctgaagatgtggcagtttattactgtcaccaggcttatagttctccgtacacgtttggccaggggaccaagctggagatcaaa

SEQ ID NO: 34 - protein, 107 aa, Artificial Sequence
  4B6 VL
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 35 - protein, 327 aa, Artificial Sequence
  huIgG4mt10 pro
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK